(19) United States
(12) Patent Application Publication
(10) Pub. No.: US 2023/0400460 A1
(43) Pub. Date: Dec. 14, 2023

(54) COMPUTER IMPLEMENTED METHOD FOR ANALYZING HOST PHAGE RESPONSE DATA

(71) Applicant: Adaptive Phage Therapeutics, Inc., Gaithersburg, MD (US)

(72) Inventors: Rob COHEN, Kensington, MD (US); Miguel BARRETO-SANZ, Lausanne (CH); Priyesh AGRAWAL, Philadelphia, PA (US)

(21) Appl. No.: 18/454,156

(22) Filed: Aug. 23, 2023

Related U.S. Application Data
(63) Continuation of application No. PCT/US22/17430, filed on Feb. 23, 2022.
(60) Provisional application No. 63/153,039, filed on Feb. 24, 2021.

(51) Int. Cl. G01N 33/569; G01N 33/487; G01N 33/557

(52) U.S. Cl. CPC G01N 33/56911; G01N 33/48792; G01N 33/557; G01N 2333/195

(57) ABSTRACT

A computer implemented method for analyzing host phage response data comprises fitting a sequence of sigmoidal functions at each time point from a start time to an end time and selecting the best fit at each point. The best fit over all the time points is then selected, and a search performed for a time point with a similar coefficient of determination, but closer in time to a minimum time point indicative of the end of the lag phase. The lag time for the best fit time point is then obtained from the fitted model. If the best fit fails a threshold test then the dataset is considered to be flat and the lag time is set to the end time.

### TECHNICAL FIELD

The present disclosure relates to methods for interpreting host-phage response data.

### BACKGROUND

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Multiple drug resistant (MDR) bacteria are emerging at an alarming rate. Currently, it is estimated that at least 2 million infections are caused by MDR organisms every year in the United States leading to approximately 23,000 deaths. Further, the overuse of antibiotics as well as bacteria's natural evolution will likely lead to the generation of more virulent microorganisms. Genetic engineering and synthetic biology may also lead to the generation of additional highly virulent microorganisms.

For example, Staphylococcus aureus are gram positive bacteria that can cause skin and soft tissue infections (SSTI), pneumonia, necrotizing fasciitis, and blood stream infections. Methicillin resistant S. aureus (“MRSA”) is an MDR organism of great concern in the clinical setting as MRSA is responsible for over 80,000 invasive infections, close to 12,000 related deaths, and is the primary cause of hospital acquired infections. Additionally, the World Health Organization (WHO) has identified MRSA as organisms of international concern.

In view of the potential threat of rapidly occurring and spreading virulent microorganisms and antimicrobial resistance, alternative clinical treatments against bacterial infection are being developed. One such potential treatment for MDR infections involves the use of phage. Bacteriophages (“phages”) are a diverse set of viruses that replicate within and can kill specific bacterial hosts. The possibility of harnessing phages as an antibacterial was investigated following their initial isolation early in the 20th century, and they have been used clinically as antibacterial agents in some countries with some success. Notwithstanding, phage therapy was largely abandoned in the U.S. after the discovery of penicillin, and only recently has interest in phage therapeutics been renewed.

The successful therapeutic use of phage depends on the ability to administer a phage strain that can kill or inhibit the growth of a bacterial isolate associated with an infection. Empirical laboratory techniques have been developed to screen for phage susceptibility on bacterial strains (i.e., efficacy at inhibiting bacterial growth). However, these techniques are time consuming and subjective, and involve attempting to grow a bacterial strain in the presence of a test phage. After many hours an assessment of the capability of the phage to lyse (kill) or inhibit bacterial growth is estimated (the host-phage response) by manual, visual inspection.

One such test is the plaque assay which is a semi-solid medium assay which measures the formation of a clear zone in bacterial lawn resulting from placement of a test phage and infection of the bacteria. Although the plaque assay is simple, plaque morphologies and sizes can vary with the experimenter, media and other conditions. More recently an automated high throughput, indirect liquid lysis assay system has been developed to evaluate phage growth using the OmniLog™ system (Biolog, Inc; Henry M, Biswas B, Vincent L, Mokashi V, Schuch R, Bishop-Lilly KA, Sozhamannan S. Development of a high throughput assay for indirectly measuring phage growth using the OmniLog™ system. Bacteriophage. 2012 Jul. 1; 2(3):159-167. doi: 10.4161/bact.21440. PMID: 23275867; PMCID: PMC3530525.). The OmniLog™ system is an automated plate-based incubator system coupled to a camera and computer which, using redox chemistry, employs cell respiration as a universal reporter. The wells in the plate each contain growth medium, a tetrazolium dye, a (host) bacterial strain and a phage (along with control/calibration wells). During active growth of bacteria, cellular respiration reduces a tetrazolium dye and produces a color change. Successful phage infection and subsequent growth of the phage in its host bacterium results in reduced bacterial growth and respiration and a concomitant reduction in color. The camera collects images at a plurality of time points, and each well in an image is analyzed to generate a color measure. This can be referenced to the initial color, or a reference color, so that a time series dataset of color change over time is collected (i.e., a colorimetric assay). The time series dataset for each well (i.e., host-phage combination) is graphed, and a user then (subjectively) reviews each of the graphs (e.g., 96 graphs for a 96 well plate). Interpretation of the growth curves is quite a challenging task as not only is there biological variability, but there may also be variability due to experimental sources such as medium and dye related effects which may affect interpretation of the growth curve. Thus, the user must use his/her experience, intuitive and implicit knowledge to interpret the graph and estimate the host-phage response. This leads to increased variability or quality as the interpretation is subjective and dependent on the skill level and/or the attentiveness of the user reviewing the graphs on a particular day. Further there is limited precision as most OmniLog™ systems only generate output data every 15 minutes.

Thus, there is a need to develop improved automated methods for analyzing/interpreting host phage response data, for example to reduce variability based on a human's interpretation, or to at least provide a useful alternative to existing methods.

### SUMMARY

According to a first aspect, there is provided a computer implemented method for analyzing host phage response data, the method comprising:
receiving or accessing a host phage response dataset, wherein the dataset comprises a time series dataset for a host-phage combinations in which a host bacteria is grown in the presence of a phage, and each data point in the time series dataset associated comprises a measurement of a parameter indicative of the growth of the host bacteria in the presence of the phage at a specific time;
obtaining an estimate of a lag time comprising:
   for a plurality of time points in a time window from an initial time point to an end time point fitting one or more candidate functions over a fitting time window from the initial time point to a current time point, wherein fitting estimates a set of growth curve summary parameters comprising at least a lag time and a goodness of fit parameter;
   selecting a best fit function for the current time point from the one or more fitted candidate functions based on the goodness of fit parameters, wherein the one or more candidate functions each comprise a different functional form and at least one of which is a sigmoidal function, and if none of the goodness of fit estimates pass a threshold goodness of fit then classifying the time series dataset as flat (thus demonstrating no growth) and setting the lag time to the end time point;
   selecting from the plurality of time points, a best time point based on goodness of fit values at the plurality of time points;
   searching for an alternative best time point closer in time to, and greater than, a minimum time point than the best time fit and updating the best time point to the alternative best time point if the goodness of fit of the alternative time point is within a threshold amount of the goodness of fit of the best time point; and
   estimating a lag time for the best time point, wherein if the goodness of fit exceeds a threshold goodness of fit the lag time is obtained from the growth curve summary parameter otherwise classifying the time series dataset as flat and setting the lag time to the end time point; and
reporting at least the lag time or a hold time calculated as the estimated lag time from which the lag time for a control is subtracted.

In one form, the one or more candidate functions is an ordered set of candidate functions, and fitting one or more candidate functions comprises sequentially fitting each candidate function according to the order in the ordered set and the sequential fitting is terminated and the candidate function is selected as the best fit function if the goodness of fit of the fitted candidate function exceeds a predefined threshold goodness.

In one form, the ordered set of candidate functions comprises a Gompertz function, a Logistic function and a Richards function.

In one form, if fitting of each of the Gompertz function, a Logistic function and a Richards function failed to generated a goodness of fit exceeding the predefined threshold goodness, then applying a Blackman window function to the time series data and repeating the fitting process, and if the repeated fits for each of the Gompertz function, a Logistic function and a Richards function fail to generate a goodness of fit exceeding the predefined threshold goodness, then classifying the time series dataset as flat and setting the lag time to the end time point.

In one form, prior to fitting one or more candidate functions over a fitting time window, determining a maximum height of the time series dataset, and if the maximum height is less than a threshold maximum height, then classifying the time series dataset as flat and setting the lag time to the end time point and terminating the fitting process.

In one form, searching for an alternative best time point comprises:
determining if the best time point is less than the minimum time point and if the best time point is less than the minimum time point then the alternative time point is selected based on the closest alternative time point on or after the minimum time point with a goodness of fit within a threshold difference of the goodness of fit for the best time point, and
if the best time point is greater than the minimum time point then the alternative time point is selected based on the alternative time point being greater than or equal to the minimum time point and which is the closest alternative time point to the minimum time point and with a goodness of fit within a threshold difference of the goodness of fit for the best time point.

In one form, the minimum time point is 5 hours, the goodness of fit is the coefficient of determination (R²), and the threshold difference if 0.03.

In one form, the goodness of fit is the co-efficient of determination (R²) and the predefined threshold goodness is 0.6.

In one form, the end time point is 48 hours.

In one form, the minimum time point is 5 hours.

In one form, if the time series data is classified as flat, estimating a variability measure and if the variability measure exceeds a variability threshold rejecting the time series dataset and classifying the time series dataset as abnormal.

In one form the method further comprises normalizing the time series dataset based on an associated control curve. In a further form, the host-growth curve is a host only time-series dataset and normalizing the time series dataset comprises subtracting the host only time-series dataset from the time series dataset, wherein the time series dataset and the host only time-series dataset are obtained from separate wells on the same multi-well plate. In a further form, the multi-well plate further comprises one or more media control wells and the computer implemented method further comprises performing quality assurance comprising at least identifying anomalous media control wells or anomalous host cell wells and excluding time-series datasets associated with any identified anomalous media control wells or anomalous host cell wells.

In one form, the time series dataset is obtained from a multi-well plate comprising a plurality of host-phage combinations, a set of positive control wells, a set of media control wells, a set of cell control wells, a first set of diluted host cell wells and a second set of diluted host cell wells, and the computer implemented method is performed for each host-phage combination on the multiwell plate, and a report is generated for each host-phage combination on the multiwell plate.

In one form, the growth curve summary parameters comprising at least a max height, a slope, a lag time, and an area under curve, and the goodness of fit comprises one or more of a co-efficient of determination (R²), an error term, a residual term, or a summary statistic of residuals.

In one form, the method further comprises receiving an updated host response dataset comprising additional data points and repeating the normalization obtaining and reporting steps, wherein the reporting includes an estimate of the probability that the phage is efficacious.

In one form, the method further comprises determining a classification expectancy and reporting the estimate of the probability that the phage is efficacious further comprises reporting the classification expectancy, wherein determining a classification expectancy comprises using a random forest based classifier trained on a plurality of flat host phage response datasets and a plurality of non-flat host phage response datasets and estimating the accuracy of the classifier for the plurality of time points, and reporting the classification expectancy for the current time point.

The above methods may be implemented in a non-transitory, computer program product comprising instructions to implement any of the above methods in a computing apparatus. The above methods may also be implemented in a computing apparatus comprising at least one memory and at least one processor configured to implement the above methods.

According to a second aspect, there is provided a non-transitory, computer program product comprising computer executable instructions for analyzing host phage response data, the instructions executable by a computer to:
receiving a host phage response dataset, wherein the dataset comprises a time series dataset for a host-phage combinations in which a host bacteria is grown in the presence of a phage, and each data point in the time series dataset associated comprises a measurement of a parameter indicative of the growth of the host bacteria in the presence of the phage at a specific time, normalizing the time series dataset based on an associated control curve;
obtaining an estimate of a lag time comprising:
   for a plurality of time points in a time window from an initial time point to an end time point fitting one or more candidate functions over a fitting time window from the initial time point to a current time point, wherein fitting estimates a set of growth curve summary parameters comprising at least a lag time and a goodness of fit parameter;
   selecting a best fit function for the current time point from the one or more fitted candidate functions based on the goodness of fit parameters, wherein the one or more candidate functions each comprise a different functional form and at least one of which is a sigmoidal function, and if none of the goodness of fit estimates pass a threshold goodness of fit then classifying the time series dataset as flat and setting the lag time to the end time point;
   selecting from the plurality of time points, a best time point based on goodness of fit values at the plurality of time points;
   searching for an alternative best time point closer in time to, and greater than, a minimum time point than the best time fit and updating the best time point to the alternative best time point if the goodness of fit of the alternative time point is within a threshold amount of the goodness of fit of the best time point; and
   estimating a lag time for the best time point, wherein if the goodness of fit exceeds a threshold goodness of fit the lag time is obtained from the growth curve summary parameter otherwise classifying the time series dataset as flat and setting the lag time to the end time point; and
reporting at least the lag time or a hold time calculated as the estimated lag time from which the lag time for a control is subtracted.

According to a third aspect, there is provided a computing apparatus comprising:
at least one memory, and
at least one processor wherein the memory comprises instructions to configure the processor to:
receiving a host phage response dataset, wherein the dataset comprises a time series dataset for a host-phage combinations in which a host bacteria is grown in the presence of a phage, and each data point in the time series dataset associated comprises a measurement of a parameter indicative of the growth of the host bacteria in the presence of the phage at a specific time;
normalizing the time series dataset based on an associated control curve;
obtaining an estimate of a lag time comprising:
   for a plurality of time points in a time window from an initial time point to an end time point fitting one or more candidate functions over a fitting time window from the initial time point to a current time point, wherein fitting estimates a set of growth curve summary parameters comprising at least a lag time and a goodness of fit parameter;
   selecting a best fit function for the current time point from the one or more fitted candidate functions based on the goodness of fit parameters, wherein the one or more candidate functions each comprise a different functional form and at least one of which is a sigmoidal function, and if none of the goodness of fit estimates pass a threshold goodness of fit then classifying the time series dataset as flat and setting the lag time to the end time point;
   selecting from the plurality of time points, a best time point based on goodness of fit values at the plurality of time points;
   searching for an alternative best time point closer in time to, and greater than, a minimum time point than the best time fit and updating the best time point to the alternative best time point if the goodness of fit of the alternative time point is within a threshold amount of the goodness of fit of the best time point;
   estimating a lag time for the best time point, wherein if the goodness of fit exceeds a threshold goodness of fit the lag time is obtained from the growth curve summary parameter otherwise classifying the time series dataset as flat and setting the lag time to the end time point; and reporting at least the lag time or a hold time calculated as the estimated lag time from which the lag time for a control is subtracted.

### BRIEF DESCRIPTION OF DRAWINGS

Embodiments of the present disclosure will be discussed with reference to the accompanying drawings wherein:

FIG. 1A is a flow chart of a method for analyzing host phage response data according to an embodiment;

FIG. 1B is a flowchart of the model fitting and parameter estimation steps to obtain an estimate of a lag time of a host phage response data according to an embodiment;

FIG. 1C is a flowchart of the steps for searching for an alternative best time point in the step of finding the best time points shown in FIG. 1B according to an embodiment;

FIG. 2A is a schematic plot of the layout of a multiwell plate used to generate a plurality of host-phage response datasets according to an embodiment;

FIG. 2B is a schematic plot of the host-phage response datasets corresponding to each well in the multiwell plate of FIG. 2A according to an embodiment;

FIG. 2C is a schematic plot of the normalized host-phage response datasets shown in FIG. 2B according to an embodiment;

FIG. 3 is a schematic diagram of a computing apparatus according to an embodiment;

FIG. 4A is a plot of a flat (i.e., no growth) host-phage response dataset according to an embodiment;

FIG. 4B is a plot of a normal (i.e., a non-flat sigmoidal shape) host-phage response dataset according to an embodiment;

FIG. 4C is a series of plots of the same host-phage response dataset at different time points from 0 to 48 hours according to an embodiment;

FIG. 5A illustrates the function forms (equations) for three sigmoidal functions;

FIG. 5B is a plot of several sigmoidal functions according to an embodiment;

FIG. 5C is a plot illustrating several growth curve summary parameters according to an embodiment;

FIG. 5D is a plot of a Blackman window according to an embodiment;

FIG. 5E is a plot of the Fourier Transform of the Blackman window according to an embodiment;

FIG. 6 is a series of plots of the same host-phage response dataset at different time points from 0 to 48 hours showing the R² values of each fit according to an embodiment;

FIG. 7A is a series of plots showing different fits of the same host-phage response dataset over different fitting windows according to an embodiment;

FIG. 7B is a series of plots showing different fits of the same host-phage response dataset over different fitting windows for finding the best time point when T<5 according to an embodiment;

FIG. 8A is a series of plots of host-phage response dataset showing short lag time, medium lag time and large lag times according to an embodiment;

FIG. 8B is a color-coded matrix plot of hold times calculated from estimated lag times of host-phage response dataset for multiple phages and the same host according to an embodiment;

FIG. 8C is a color-coded matrix plot of hold times calculated from estimated lag times of host-phage response dataset for multiple phages and multiple hosts according to an embodiment;

FIG. 9A is a histogram of the estimated hold time difference between a hold time estimated by an embodiment of the method described herein, and a human estimate;

FIG. 9B is a table of the estimated hold time difference between a hold time estimated by an embodiment of the method described herein, and a human estimate used to generate FIG. 9A;

FIG. 9C is a histogram of the estimated hold time difference between a hold time estimated by an embodiment of the method described herein, and a human estimate for 695 datasets across 39 different multiwell plates; and

FIG. 10 is a set of plots showing the time taken for a machine learning model to correctly classify the efficacy of a host-phage time series dataset according to an embodiment.

In the following description, like reference characters designate like or corresponding parts throughout the figures.

### DESCRIPTION OF EMBODIMENTS

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules. “A phage formulation” can mean at least one phage formulation, as well as a plurality of phage formulations, i.e., more than one phage formulation. As understood by one of skill in the art, the term “phage” can be used to refer to a single phage or more than one phage.

The present invention can “comprise” (open ended) or “consist essentially of” the components of the present invention as well as other ingredients or elements described herein. As used herein, “comprising” means the elements recited, or their equivalent in structure or function, plus any other element or elements which are not recited. The terms “having” and “including” are also to be construed as open-ended unless the context suggests otherwise. As used herein, “consisting essentially of” means that the invention may include ingredients in addition to those recited in the claim, but only if the additional ingredients do not materially alter the basic and novel characteristics of the claimed invention.

As used herein, a “subject” is a vertebrate, preferably a mammal, more preferably a human. Mammals include, but are not limited to, murines, simians, humans, farm animals, sport animals, and pets. In other preferred embodiments, the “subject” is a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), or an ape (e.g., gorilla, chimpanzee, orangutan, gibbon). In other embodiments, non-human mammals, especially mammals that are conventionally used as models for demonstrating therapeutic efficacy in humans (e.g., murine, primate, porcine, canine, or rabbit animals) may be employed. Preferably, a “subject” encompasses any organism, e.g., any animal or human, that may be suffering from a bacterial infection, particularly an infection caused by a multiple drug resistant bacterium.

As understood herein, a “subject in need thereof” includes any human or animal suffering from a bacterial infection, including but not limited to a multiple drug resistant bacterial infection, a microbial infection or a polymicrobial infection. Indeed, while it is contemplated herein that the methods may be used to target a specific pathogenic species, the method can also be used against essentially all human and/or animal bacterial pathogens, including but not limited to multiple drug resistant bacterial pathogens. Thus, in a particular embodiment, by employing the methods of the present invention, one of skill in the art can design and create personalized phage formulations against many different clinically relevant bacterial pathogens, including multiple drug resistant (MDR) bacterial pathogens.

As understood herein, an “effective amount” of a pharmaceutical composition refers to an amount of the composition suitable to elicit a therapeutically beneficial response in the subject, e.g., eradicating a bacterial pathogen in the subject. Such response may include e.g., preventing, ameliorating, treating, inhibiting, and/or reducing one of more pathological conditions associated with a bacterial infection.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term “about” means within an acceptable error range for the particular value, such as ±1-20%, preferably ±1-10% and more preferably +1-5%. In even further embodiments, “about” should be understood to mean+/−5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Where used herein, the term “and/or” when used in a list of two or more items means that any one of the listed characteristics can be present, or any combination of two or more of the listed characteristics can be present. For example, if a composition is described as containing characteristics A, B, and/or C, the composition can contain A feature alone; B alone; C alone; A and B in combination; A and C in combination; B and C in combination; or A, B, and C in combination.

The term “phage sensitive” or “sensitivity profile” means a bacterial strain that is sensitive to infection and/or killing by phage and/or in growth inhibition. That is phage is efficacious or effective in inhibiting growth of the bacterial strain.

The term “phage insensitive” or “phage resistant” or “phage resistance” or “resistant profile” is understood to mean a bacterial strain that is insensitive, and preferably highly insensitive to infection and/or killing by phage and/or growth inhibition. That is phage is not efficacious or is ineffective in inhibiting growth of the bacterial strain.

A “therapeutic phage formulation”, “therapeutically effective phage formulation”, “phage formulation” or like terms as used herein are understood to refer to a composition comprising one or more phage which can provide a clinically beneficial treatment for a bacterial infection when administered to a subject in need thereof.

As used herein, the term “composition” encompasses “phage formulations” as disclosed herein which include, but are not limited to, pharmaceutical compositions comprising one or more purified phage. “Pharmaceutical compositions” are familiar to one of skill in the art and typically comprise active pharmaceutical ingredients formulated in combination with inactive ingredients selected from a variety of conventional pharmaceutically acceptable excipients, carriers, buffers, and/or diluents. The term “pharmaceutically acceptable” is used to refer to a non-toxic material that is compatible with a biological system such as a cell, cell culture, tissue, or organism (or at least non-toxic in amounts typically used). Examples of pharmaceutically acceptable excipients, carriers, buffers, and/or diluents are familiar to one of skill in the art and can be found, e.g., in Remington's Pharmaceutical Sciences (latest edition), Mack Publishing Company, Easton, Pa. For example, pharmaceutically acceptable excipients include, but are not limited to, wetting or emulsifying agents, pH buffering substances, binders, stabilizers, preservatives, bulking agents, adsorbents, disinfectants, detergents, sugar alcohols, gelling or viscosity enhancing additives, flavoring agents, and colors. Pharmaceutically acceptable carriers include macromolecules such as proteins, polysaccharides, polylactic acids, polyglycolic acids, polymeric amino acids, amino acid copolymers, trehalose, lipid aggregates (such as oil droplets or liposomes), and inactive virus particles. Pharmaceutically acceptable diluents include, but are not limited to, water, saline, and glycerol.

As used herein, the term “estimating” encompasses a wide variety of actions. For example, “estimating” may include calculating, computing, processing, determining, deriving, investigating, looking up (e.g., looking up in a table, a database or another data structure), ascertaining and the like. Also, “estimating” may include receiving (e.g., receiving information), accessing (e.g., accessing data in a memory) and the like. Also, “estimating” may include resolving, selecting, choosing, establishing and the like.

Further, as used herein, the test bacteria may be provided to the wells “free” in a liquid preparation (e.g., as a planktonic preparation) or provided as a biofilm. If a biofilm is provided, then the methods can be tested on a single phage or can also be used to test phage/phage synergy, phage/antibiotic synergy, and even phage/phage/antibiotic synergy by just modifying the starting materials. In preferred aspects, lysis of a bacterium by a phage can be measured using assays known in the art, such as but not limited to (a) growth inhibition (either grown as planktonic cells or within a biofilm); (b) optical density; (c) metabolic output; (d) photometry (e.g., fluorescence, absorption, and transmission assays); and/or (e) plaque formation. Phage activity, including synergy of the phage—whether it be phage-phage synergy or phage-antibiotic synergy—can be measured using methods known in the art or described in U.S. Applications 63/153,039, 63/208,173, 63/246,601, 63/291,955, 63/310,875, and 63/288,793, each of which are herein incorporated by reference in their entirety.

For example, those skilled in the art will appreciate that a biofilm of a bacteria may be prepared in vitro by culturing a suitable bacteria (which may be a bacterial strain obtained from a patient sample) in the presence of a solid surface, such as provided by, for example, a polymeric film or planar surface, or a particle or bead comprised of a polymeric material or other inert material such as glass. In some embodiments, the test bacteria is preferably provided to the test wells in the form of a biofilm attached to the surface of a bead such as a glass bead with a diameter of about 1 to 5 mm, and preferably, a glass bead of about 4 mm (which are suitable for, for example, conventional 96-well multiwell plates, wherein one biofilm-coated bead per test may be sufficient). Otherwise, biofilm produced through in vitro culture may, for example, be removed from a surface (if necessary) and processed into consistently-sized pieces (e.g. 2 mm×2 mm substantially planar pieces) by any method that would be apparent to those skilled in the art (e.g. vigorous agitation and/or microdissection).

Embodiments of computer implemented methods and systems for analyzing host phage response datasets will now be described.

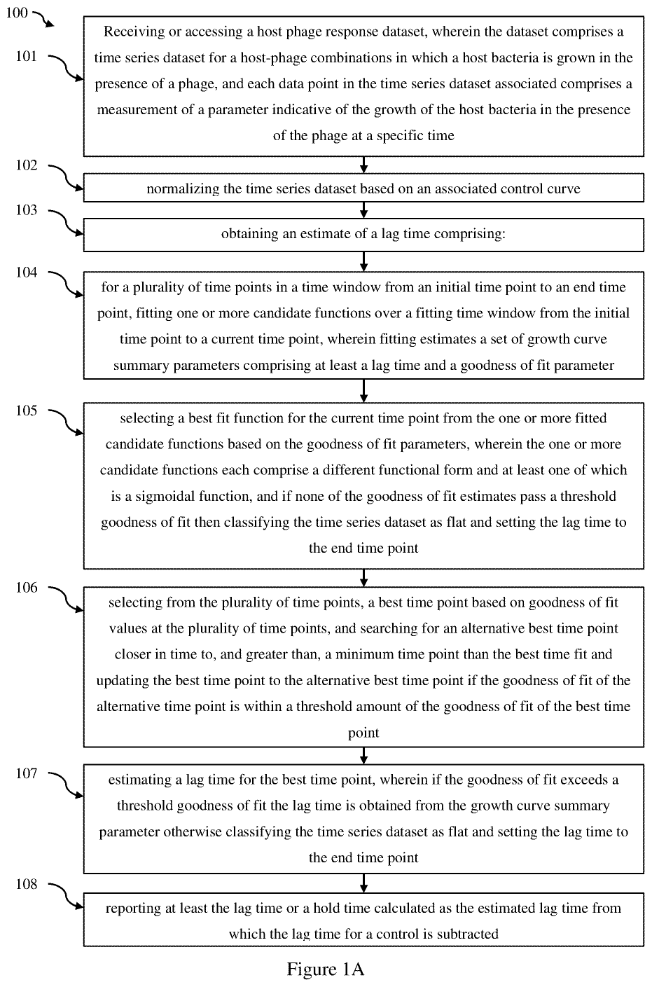

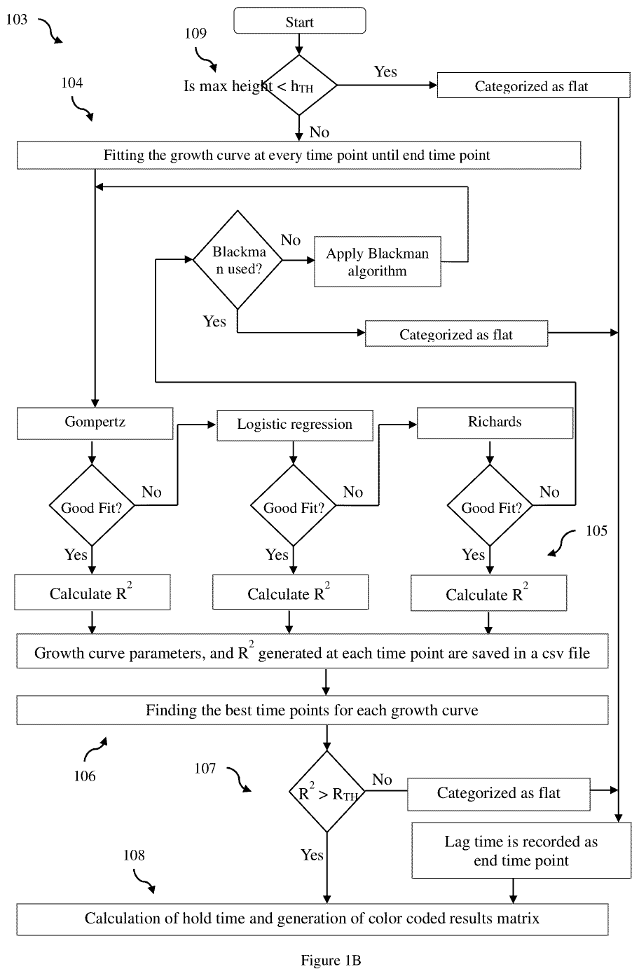

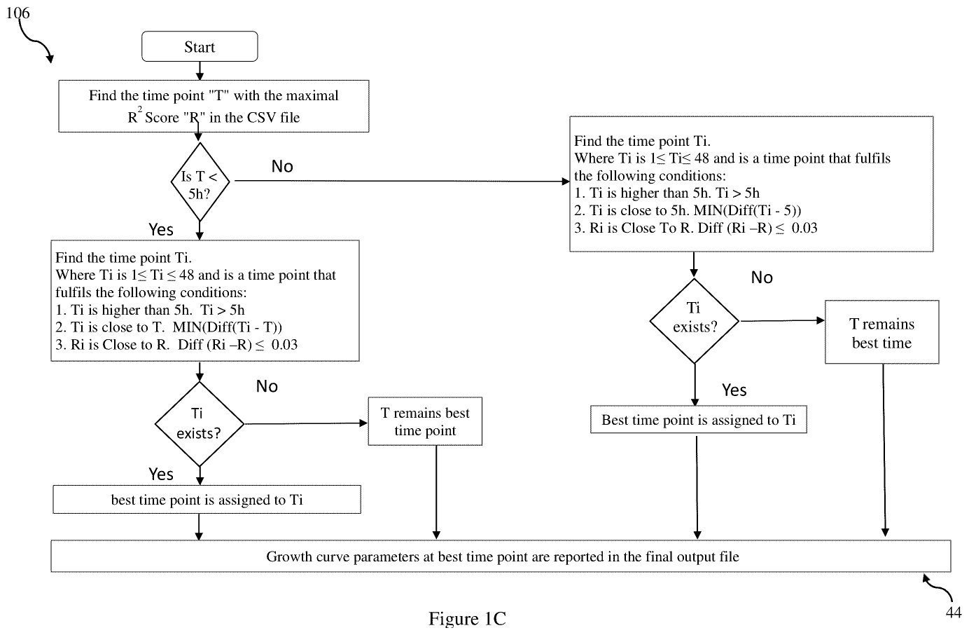

FIG. 1A is a flow chart of a computer implemented method 100 for analyzing host phage response data according to an embodiment. A host phage response dataset is received or accessed by the computing apparatus 101. This may be sent by the apparatus (e.g., OmniLog™ or Biotec™) which generated the dataset, or a computing apparatus associated with the apparatus (e.g., a control computer), or the data may be accessed from a storage device. The dataset comprises a time series dataset for a host-phage combination in which a host bacterium is grown in the presence of a phage. Each data point in the time series dataset associated comprises a measurement of a parameter indicative of the growth of the host bacteria in the presence of the phage at a specific time.

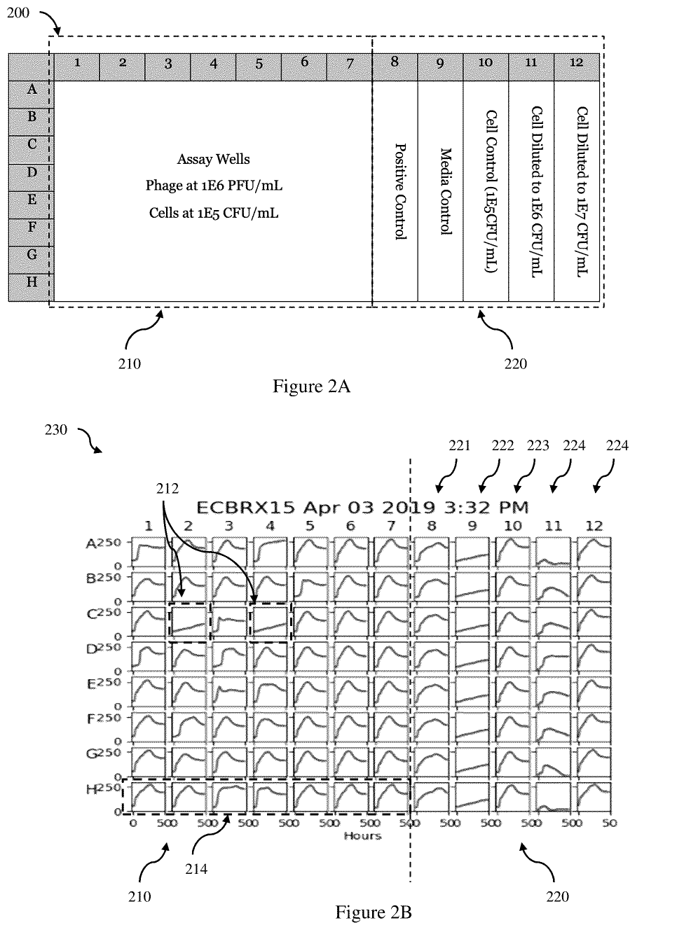

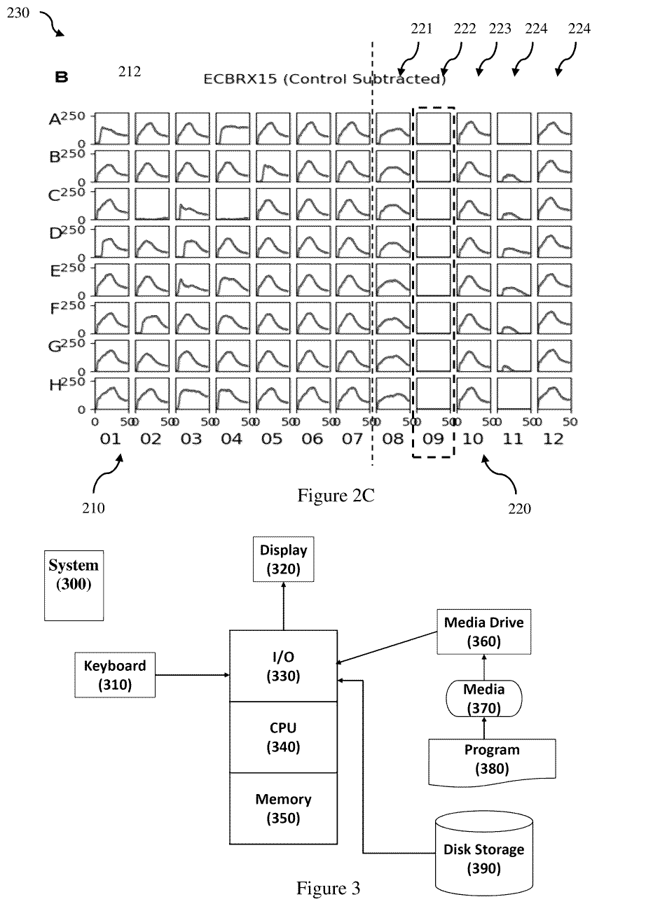

The data may be provided an electronic format, such as a CSV input file exported by an OmniLog™ (or similar host response, such as Biotec™) apparatus. FIG. 2A is a schematic plot of the layout 200 of a multiwell plate (e.g., 96 wells) used to generate a plurality of host-phage response datasets according to an embodiment. FIG. 2B is a schematic plot of the host-phage response datasets corresponding to each well in the multiwell plate of FIG. 2A according to an embodiment. This shows two good phage 212 and poorly performing phage 214. These are arranged in a 7×8 grid of assay wells 210 (rows A to H and columns 1-12) and a set of control wells 220 comprising positive control wells 221, media control wells 222, cell control wells 223, and two cell dilutions 224, 225. The media control wells 222 in column 9 contain just growth media to allow detection of possible contamination. The cell control wells in column 10 comprise bacteria grown in media in the absence of phage and can be used to detect contamination or other growth issues. In some cases, the same host bacteria are grown in each well of a row with different phage in each column. In some cases, the same host bacteria may be grown in each well of the plate (apart from media controls). In some cases there may be a mix and match of bacteria and phage in wells (e.g. 4 different bacteria and 9 different phage). A master phage plate file (in csv format) may also be provided which contains well-wise phage information. This file is used for the annotation of the final results. Other metadata relating to the experimental run may also be provided such as operator's name, project ID, master phage plate ID and date of experiment.

Once the data is received normalization and quality control/quality assurance may be performed 102. For example, as shown in FIG. 2B, the media control wells 222 in column 9 have a general linear increase over time. Thus, in one embodiment a normalization step 102 is performed in which each time series dataset is normalized based on an associated control curve. For example, for each dataset in columns 1-7 of a row, the media control curve 221 in column 9 of the same row is subtracted. FIG. 2C is a schematic plot of the normalized host-phage response datasets shown in FIG. 2B.

Other quality assurance checks may also be made to identify anomalous media control wells 222 or anomalous host cell only wells 223. Datasets associated with any identified anomalous media control wells or anomalous bacterial host cell only wells may be excluded. For example, an entire row may be discarded if the host growth curve in cell control well (column 10) is flat or has an unusual shape such as non-sigmoidal or with unusual lag times which may indicate contamination or other growth issue with the bacteria host. For example, E. coli typically has a lag time in the range of 3-5 hours, and thus a lag time outside of this range may indicate an issue with the bacteria. Various rule based or threshold-based quality assurance procedures may be used. Further a machine learning method may also be used to perform quality assurance. This may be trained on labelled control wells 220 and used to identify anomalous control wells.

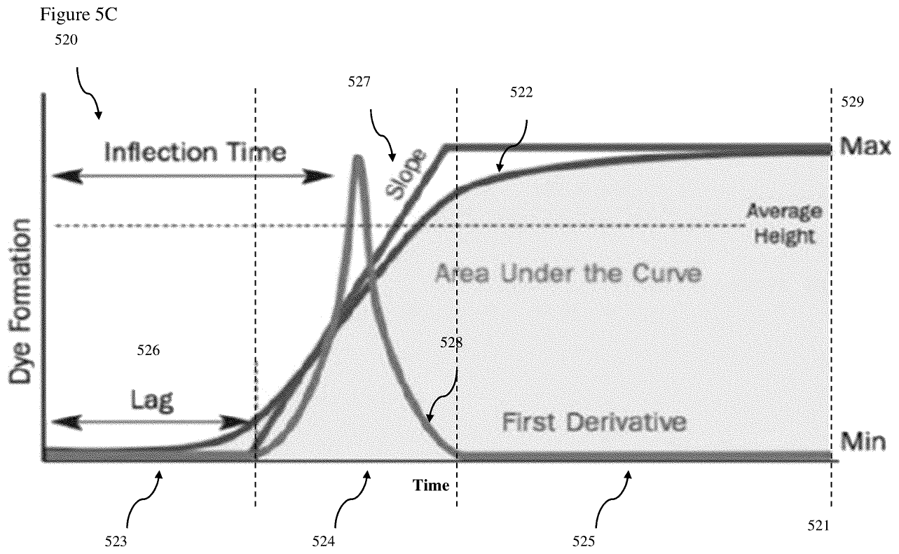

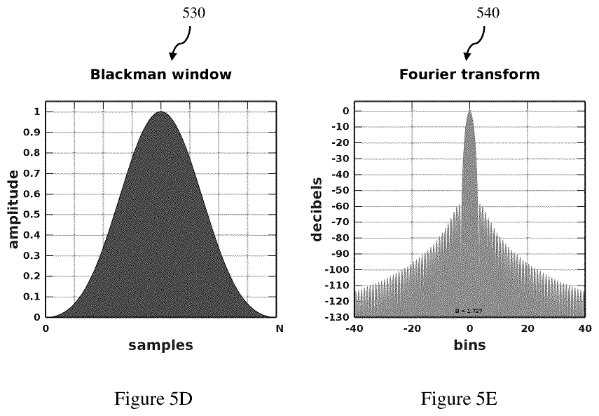

Once any normalization and quality assurance has been performed, automated model fitting and parameter estimation may then be performed, for example to obtain an estimate of the lag time for each dataset 103, along with other model parameters that characterize the curve. A typical growth curve 522 is shown in FIG. 5C has an S or sigmoidal shaped profile with a lag phase 523, growth phase 524 and stationary phase 525 up to end time point 521. The lag phase 523 can be characterized by a lag time 526 during which there is minimal observable growth of the bacteria—that is the curve is substantially flat. During the growth phase 524 the bacteria grow typically at a reasonably constant growth rate, until the growth begins to stabilize, and the curve flattens into the stationary (or stabilization) phase 525 where it asymptotically approaches a maximum height 529. The growth phase 528 can be characterized as following an approximately linear path with rolled off ends at the transition between the adjacent phases, with the linear growth characterized by a constant slope 527. The first derivative of the curve 528 can be used to delineate the phase transitions. The lag time can be defined as the time interval from the starting time to the time point where the growth curve starts to show evidence of an increase. It may be defined in a statistical model or could be set as time point when some threshold value or condition is satisfied or met. For example, it could be the first time point where the observed value is equal to some percentage of the maximum value, such as 1%, 2%, 5% or 10%. Alternatively, it could be defined based on a large deviation, such as the first value exceeding a measure variability (e.g., 2, 3 or 5 standard deviations calculated over a time window since the start of the experiment. In another embodiment, the lag time could be the intercept between the horizontal baseline starting level and the tangent line to maximum growth rate during the exponential phase of the growth curve (on a log scale). Statistical models, such as ANOVA and a may also be used to estimate the lag time, for example by using a model to estimate the best fit baseline values and growth rates in order to estimate the intercept point. The lag time may be used to calculate a hold time which is the lag time from which the lag time for a control is subtracted, thus representing a measure of deviation from a standard lag time.

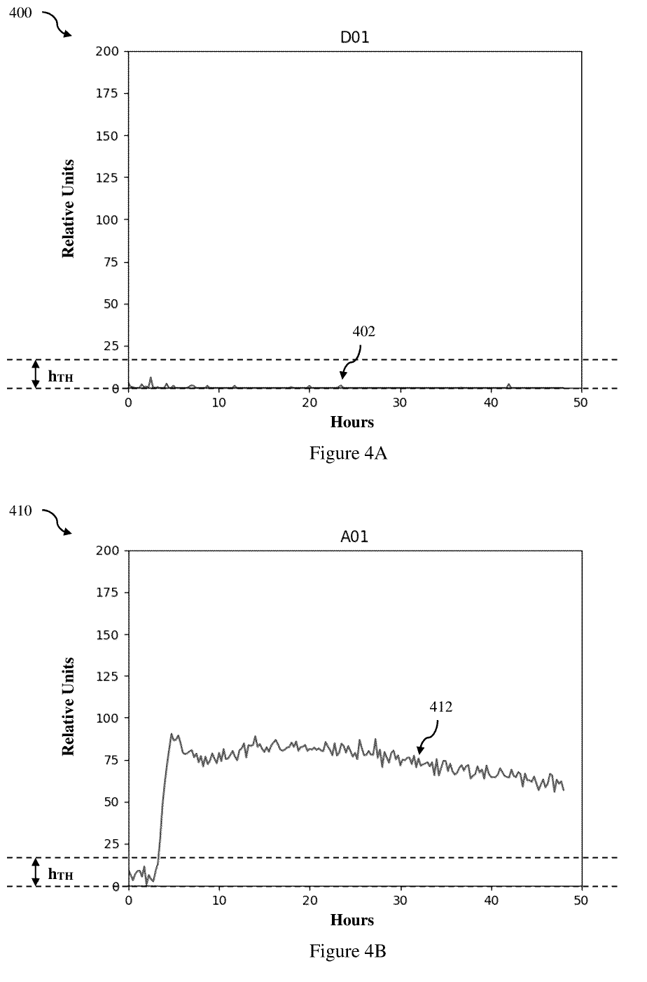

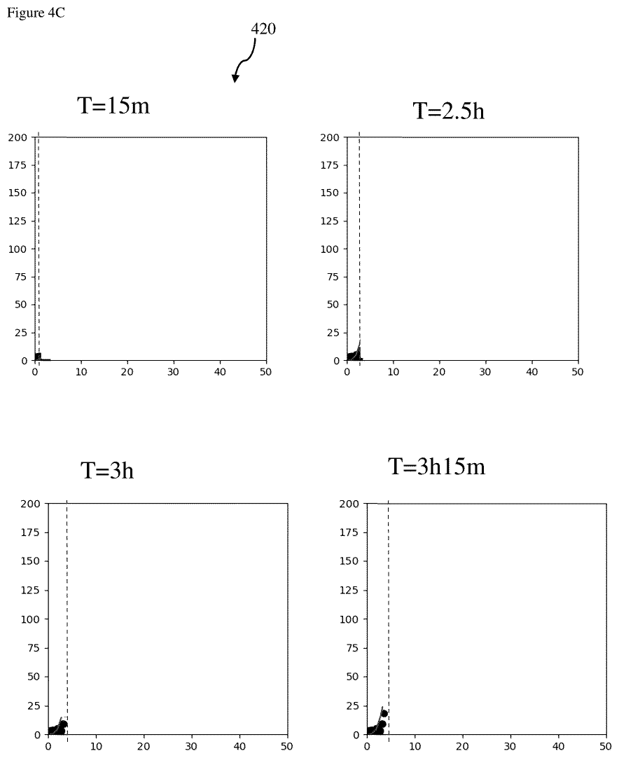

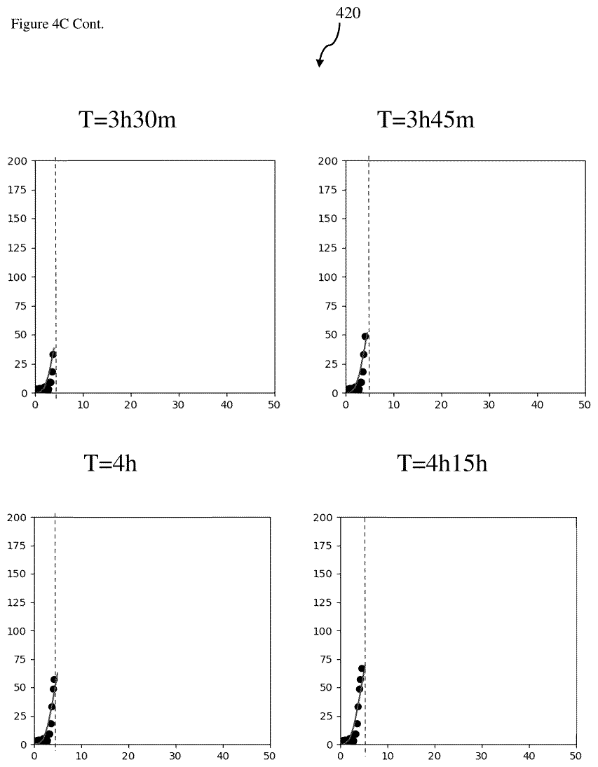

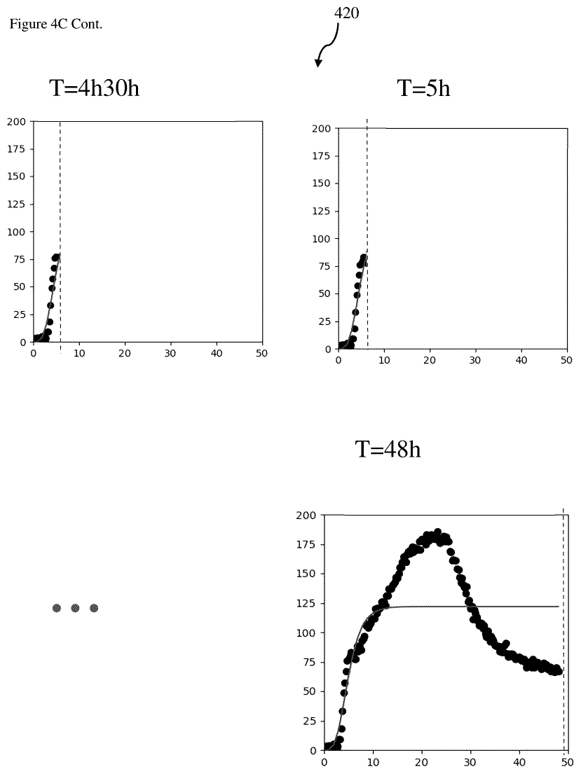

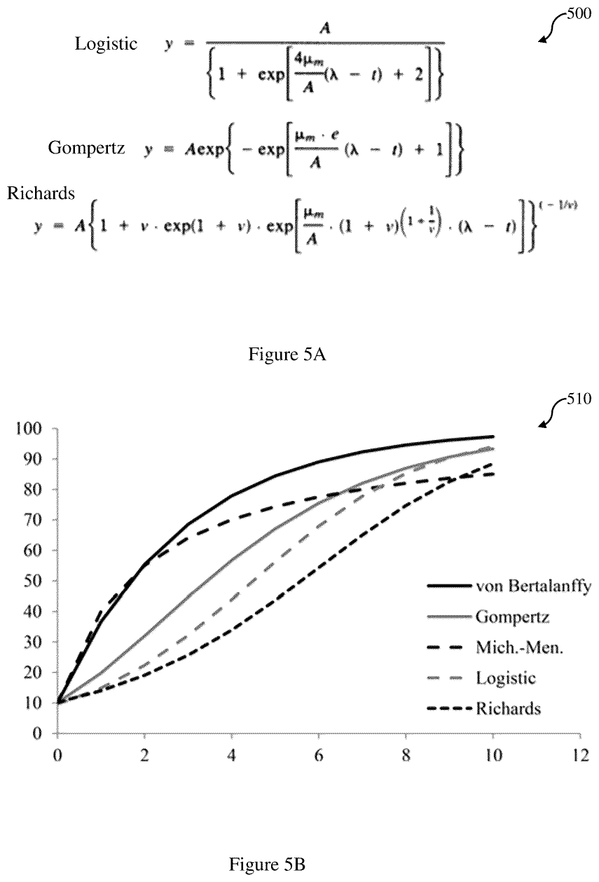

In the case that a phage inhibits the growth of the host bacteria, the growth curve will have a substantially flat profile (i.e., is not sigmoidal) which can be characterized as having a lag time 254 equal to the end time. This is shown in FIG. 4A which is a plot of a flat host-phage response dataset 402 according to an embodiment. For comparison FIG. 4B is a plot of a normal (non-flat) host-phage response dataset 412 according to an embodiment.

Thus, a preliminary step 109 may comprise determining the maximum height 529 of the time series dataset, and if the maximum height is less than a threshold maximum height h_{TH}, then classifying the time series dataset as flat and setting the lag time 524 to the end time point 521 and terminating (or bypassing) the fitting process. The threshold maximum height h_{TH} may be determined from the analysis of historical growth curves obtained using the OmniLog™ or similar platform (e.g., Biotec™). The threshold h_{TH} is used for identifying the flat growth curves and is determined automatically by the algorithm by looking at the corresponding cell control. h_{TH} is defined as the 30% of the maximum density of the cell control. If an assay well has reached its maximum density which is less than 30% of its cell control, it is marked as a flat curve by the algorithm. The threshold may be varied based on the specific equipment used to generate the host phage growth curves.

The model fitting 104 is performed for a plurality of time points in a time window from an initial time point to an end time point 529. For example, this may be from the start time (0 h) to an end time (24, 36, 48 hours etc.). The end time point may be arbitrarily set to a time point sufficient for growth of the host bacteria to have stabilized and may be set prior to the end of the dataset. For example, an experiment may run for 50 hours but the end time point may be set to 48 hours (or earlier such as 24 or 36 hours). The plurality of time points may be every time point in the dataset, at periodic intervals (e.g., every 15 minutes) or uniformly across the dataset. The interval between time points at which fitting is performed need to not be constant though (for example, some points could be lost due to data corruption, or the apparatus may not sample the wells at precisely regular intervals. For example, a fully loaded OmniLog™ machine takes around 15 minutes to sample every well in every plate, and thus data for each well may be collected at approximately 15 minutes intervals.

At each time point we fit one or more candidate functions over a fitting time window 105. The fitting time window may be from the initial time point (i.e., start time of the assay) to the current time point. The fitting fits a model of the candidate function to estimate a set of growth curve summary parameters (the fitted model parameters) comprising at least a lag time and a goodness of fit parameter. As shown in FIG. 5C, the model may fit parameters such as a max height 529, a slope 527, a lag time 526, and an area under curve. The goodness of fit parameter measures how well the candidate function (or the model of the candidate function) matches the observed dataset and typically is a parameter that summarizes the difference between the observed value and the expected value from the model. In some embodiments the goodness of fit is the co-efficient of determination (R²), although others may be used such as a parameter based upon an error term or a residual term, or a summary statistic of residuals.

This process is further illustrated in FIG. 4C which is a series of plots of the same host-phage response dataset at different time points from 0 to 48 hours (end time 529). Each plot shows the data points from 0 to the current time, with the vertical dashed line representing the current time (listed in the plot title) and solid red line representing the model fit from the start time to the current time.

It can be seen in the plot at 48 hours the data after 10 hours has a peaked profile whilst the fitted line is flat. Often in host-phage growth curves the stationary phase 525 is not particularly flat due to media and dye related effects and during the stationary phase the observed data is not always a true measure of growth. This generates considerable variability from plot to plot or experiment to experiment, making automated model fitting challenging.

To cope with the inherent variability of host-phage growth curves, embodiments of the model fitting method thus fit one or more candidate functions at each time point, and we select a best fit function for the current time point from the one or more fitted candidate functions based on the goodness of fit parameters 105. Each of candidate functions comprise a different functional form, at least one of which is a sigmoidal function (i.e., has an S shaped form). The sigmoidal functions may be a Gompertz function, a Logistic function or a Richards function, the functional forms 500 of which are shown in FIG. 5A. A comparative plot 510 of several sigmoidal functions with different functional forms is shown in FIG. 5B. Other candidate functions such as a third order or fifth order polynomial fit, linear models (including generalized linear models, non-linear regression models, may also be used. The candidate functions have different functional forms to capture different curve shapes, and deviations from a pure growth curve to capture the observed variability due to various experimental effects (e.g., dye and media effects). If none of the goodness of fit estimates pass a threshold goodness of fit (e.g., R²<0.6) then we classify the time series dataset as flat and set the lag time to the end time point 529 and cease model fitting for this dataset—see FIG. 2A.

FIG. 2A shows a flowchart of an embodiment in which fitting one or more candidate functions comprises sequentially fitting each candidate function according to the order in an ordered set of candidate functions. After fitting each model the goodness of fit of the fitted candidate function is evaluated to determine if the model is a good fit, for example by testing if the goodness of fit exceeds a predefined threshold goodness (e.g. R²>0.6), and if the model is considered a good fit the sequential fitting is terminated and the candidate function is selected as the best fit function for this time point and the model growth curve parameters, R² and other data are stored, such as by saving to a CSV (comma-separated values) file.

In this embodiment the sequence is a Gompertz function followed by a Logistic function, followed by a Richards function. If neither of these functions generates a good fit, we then apply a Blackman window function to the time series data and repeat the fitting process. FIG. 5D shows a plot 530 of a Blackman window which has a maximum value at a peak point function and is zero outside of some range, so that when applied to the time series data, data outside of the range is forced to zero to restrict the effect of distant points in the curve fitting. FIG. 5E is a plot 540 of the Fourier Transform of the Blackman window according to an embodiment showing the suppression of side lobes. Other window functions may be used such as generalized cosine windows, Gaussian, sine, B spline etc. If the repeated fits for each of the Gompertz, Logistic and Richards functions fail to generate a goodness of fit exceeding the predefined threshold goodness, then we classify the time series dataset as flat and set the lag time 526 to the end time point 540. This is shown by the check for if a Blackman has already been used in FIG. 2A.

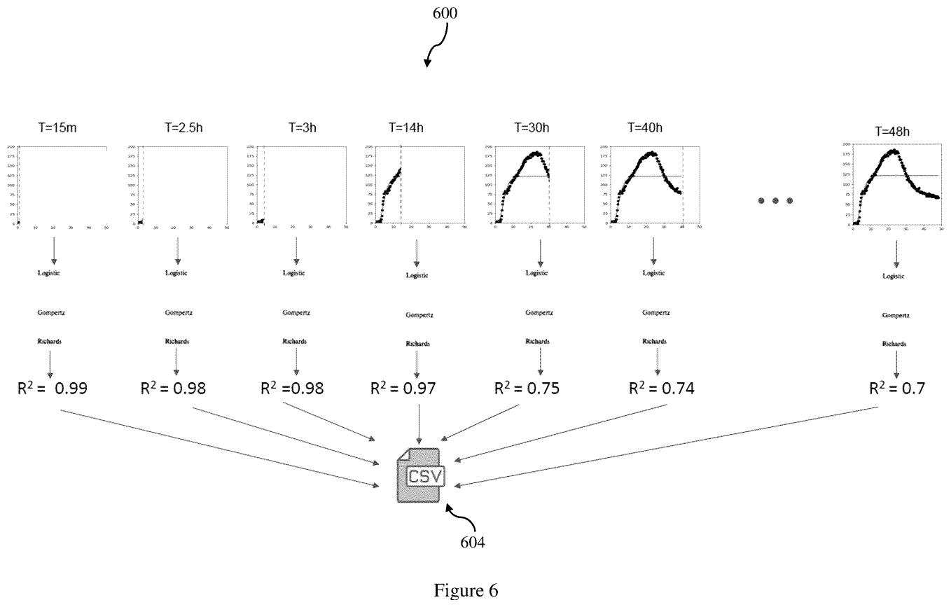

Having obtained a curve fit for each of the time points in the dataset (or at least a plurality of time points in the dataset) along with a goodness of fit measure such as R² the data may be stored or written to a file, such as a CSV file 604 as illustrated in FIG. 6 which shows a series of plots of the same host-phage response dataset at different time points from 0 to 48 hours showing the R² values of each fit.

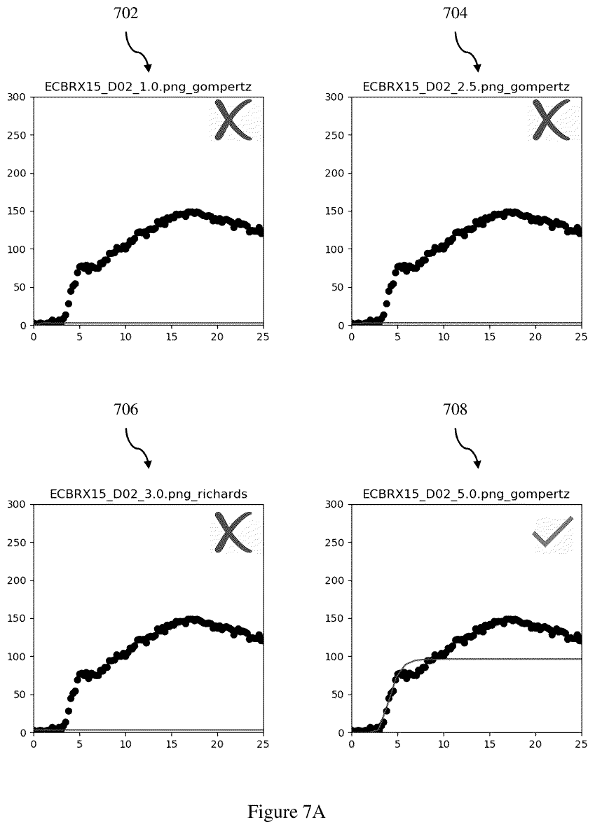

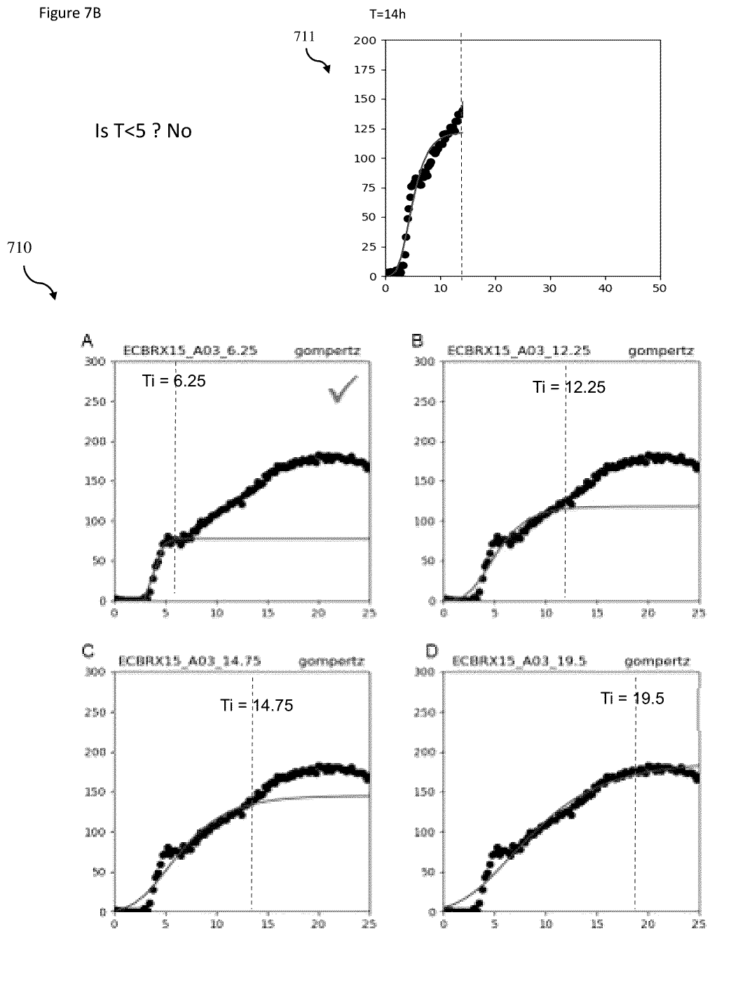

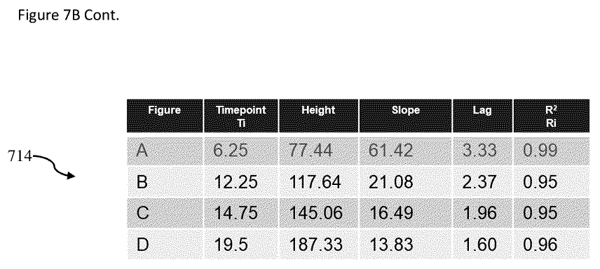

We then select a best time point based on goodness of fit values at the plurality of time points 106. A flowchart of a search for the best time point is further illustrated in FIG. 1C, and further illustrated in FIGS. 7A to 7C search for the best time point is further illustrated in FIG. 1C, and further illustrated in FIGS. 7A to 7C. We start by initially searching for the time point with the largest R² value from the stored results (e.g., the CSV file or data structure in memory) and select this time point T as the best time point (or at least the initial best time point). To ensure the best time is robustly selected, we may perform an additional search for an alternative best time point closer in time to, and greater than, a minimum time point Tₘᵢₙ than the best time fit. If such an alternative time point is found, we then update the best time point to the alternative best time point. This may be based on selection criteria which require that the goodness of fit of the alternative time point is within a threshold amount of the goodness of fit of the best time point. This search effectively tries to bias the search towards the best fit around the growth phase.

In one embodiment the minimum time point Tₘᵢₙ is set at 5 hours. Most bacterial, in the absence of phage, have lag times of 3 hours to 5 hours, and thus the minimum time represents a time around or after the end of the lag phase. Accordingly in some embodiments Tₘᵢₙ is set at a time between 3 and 6 hours, such as 3, 3.5, 4, 4.5, 5, 5.5 or 6 hours, with the specific value used based on analysis of historical growth curves for the relevant host bacteria or multiple bacteria in which some average or central value may be selected. In the following exemplary embodiments the minimum time point Tₘᵢₙ is set at 5 hours.

As shown in FIG. 7A, at very low time points (i.e., <5H) many fit models can display a good fit, since those were the regions where the curves are mostly flat. For example, as shown in FIG. 7A, the model (red) in the first three plots in which fitting time periods are 1 hour 702, 2.5 hours 704 and 3 hours 706 all show high similarity with actual growth data (black), and thus all these models will produce a high R² score in this region. For example, R²=0.98 for the 3-hour case 706. However, selection of these time points would give a false positive Model Fit if used. To prevent this the algorithm forces a search of models with time fit periods above 5 hours. Plot 708 shows the model fit for a time point of 5 hours, and it can be seen the model (in red) is a better fit to the data than the previous three.

As shown in FIG. 1C we can define selection criteria for selecting an alternative time point Tᵢ. This may comprise that the alternative time point Tᵢ minimum time point (Tₘᵢₙ e.g., 5 hours), and that the goodness of fit of the alternative time point is within a threshold amount of the goodness of fit of the best time point, e.g. |Rᵢ²−R²|≤0.03, and that we select the closest alternative time point to either the minimum time point or the current time point e.g., use min (|Tᵢ−Tₘᵢₙ|) or min(|Tᵢ−T|). Either the minimum time point or the current time point can be used as T and Tₘᵢₙ are constant for the search, and both are less than Tᵢ e.g. T<Tₘᵢₙ<Tᵢ. The first condition ensures the alternative time point is in a desired range (i.e., >5 hours) and the second condition allows selection of all time points which have R² close to the best R² (in the desired range). This allows us to trade off some statistical performance for an earlier time point. The third condition defines that if we have multiple possible alternative time points, choose the earliest (i.e. closest to 5 hours). If such an alternative time point is found we update the current time best point to the alternative time point (T=Tᵢ), otherwise we keep the current best time point T. Thus, we force a search for a good fit closer to the growth phase. Thus, in one embodiment the minimum time point is selected based on the typical (historical) start or midpoint of the growth phase.

If, however the best time point is greater than the minimum time point then the alternative time point is selected based on the alternative time point being greater than or equal to the minimum time point and which is the closest alternative time point to the minimum time point with a goodness of fit within a threshold difference of the goodness of fit for the best time point. This is to avoid the model fit focusing (or being biased by) on late times in the stationary phase where the curve may be flat and well fit at the expense of well-fitting at the growth phase. As shown in FIG. 1C we can define selection criteria for selecting an alternative time point Tᵢ. This may comprises that the alternative time point Ti>minimum time point (e.g. 5 hours), that goodness of fit of the alternative time point is within a threshold amount of the goodness of fit of the best time point, e.g. |Rᵢ²−R²|≤0.03, and that we select the closest alternative time point to the minimum time point, e.g. min (|Tᵢ−Tₘᵢₙ|). If such an alternative time point is found we update the current time best point to the alternative time point (T=Tᵢ), otherwise we keep the current best time point T.

This is illustrated in FIG. 7B which shows a plot of the initial best fit time point of 14 hours model 711 along with fits for fitting time windows of 6.25, 12.25 14.75 and 19.5 hours in plots A-D, along with the model fit parameters in table 714. This search identifies an alternative time point Ti at 6.25 hours (plot A).

In the above embodiment, the minimum time point is 5 hours, the goodness of fit is the coefficient of determination (R²), and the threshold difference if 0.03.

We then estimate a lag time for the best time point 107. Additionally, a test is performed to determine if the goodness of fit exceeds a threshold goodness of fit. In one form, the goodness of fit is the co-efficient of determination (R²) and the predefined threshold goodness is 0.6 (e.g., R²>0.6). In that case the lag time is obtained from the growth curve summary parameter otherwise we classify the time series dataset as flat and set the lag time 523 to the end time point 529. We then report at least the lag time or a hold time calculated as the estimated lag time from which the lag time for a control is subtracted 108.

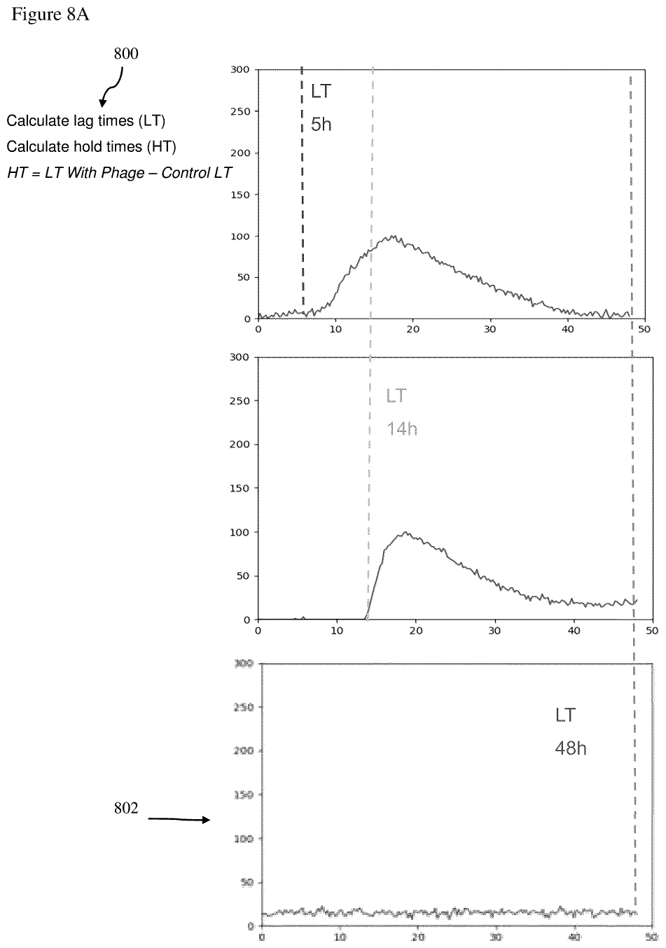

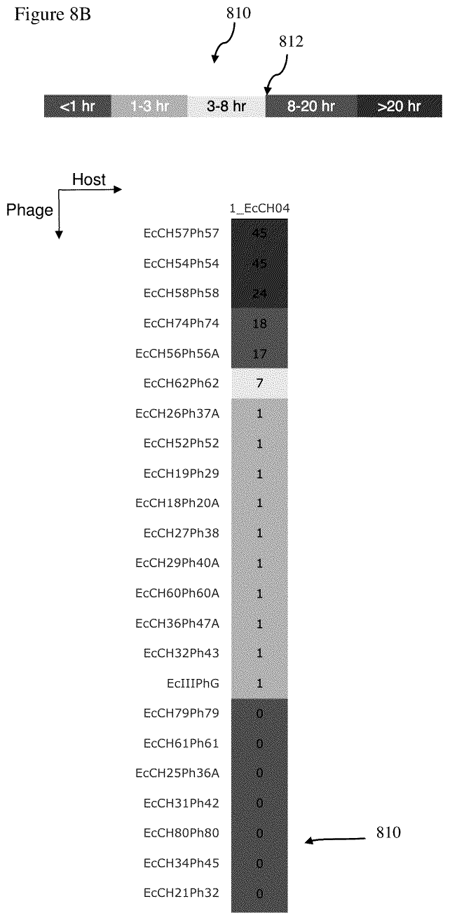

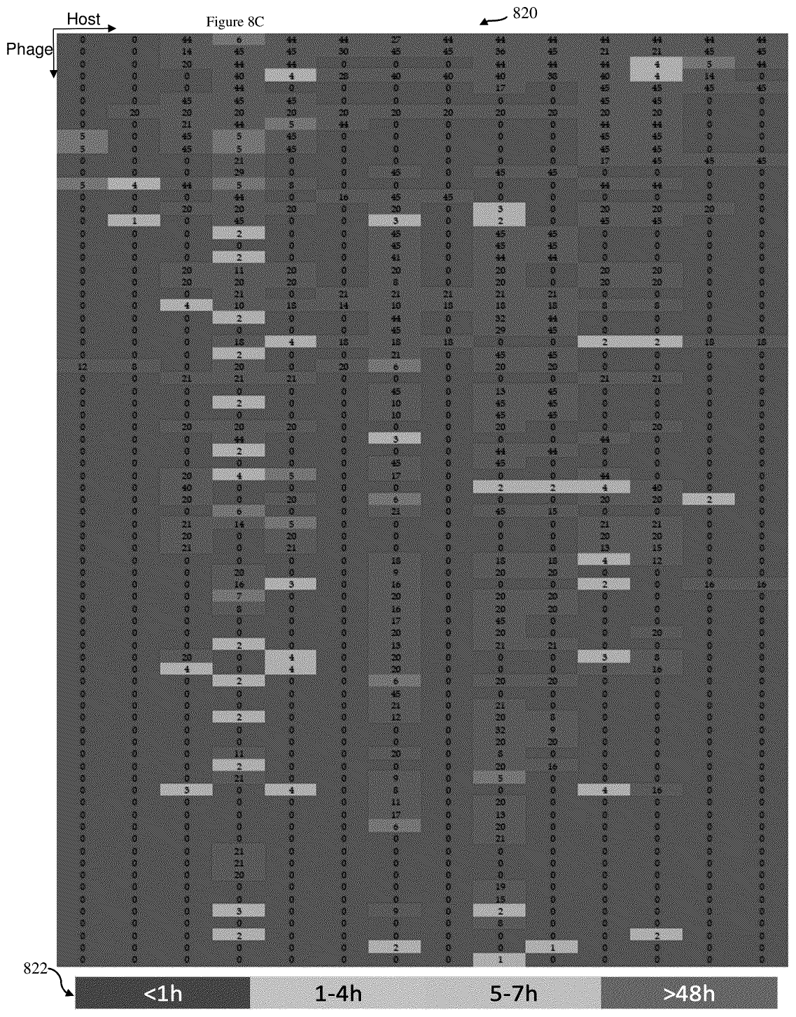

This is further illustrated in FIG. 8A which is a series of plots of host-phage response dataset showing short lag time (5 h), medium lag time (14 h) and large lag times (48 h) according to an embodiment. The hold time can be calculated from a control phage lag time (i.e., hold time=lag time with phage−control lag time). FIG. 8B is a color-coded matrix plot of hold times calculated from estimated lag times of host-phage response dataset for multiple phages and the same host according to an embodiment.

The legend 812 defines colors for each of a plurality of time windows (<1 hour (red), 1-3 hours (orange), 3-8 hours (yellow), 8-20 hours (green) and >20 hours (blue)). Phage which inhibits bacterial growth (i.e., good phage) have large hold times (>20 hours) and ineffective phage (i.e., poor phage) have short hold times (<1 hour). FIG. 8C is another plot of a color-coded matrix 820 of hold times calculated from estimated lag times of host-phage response dataset for multiple phages (rows) and multiple hosts (columns), using another legend 822 according to an embodiment. In this plot the legend 822 comprises 4 time windows: <1 hour (red); 1-4 hours (orange); 5-7 hours (green); and >8 hours (blue). The report (which may be provided by a webserver) may include plots such as those illustrated in FIGS. 8B and 8C to report results to users to allow selection of phage for treatment of patients. That is phage in blue cells may be selected for treatment.

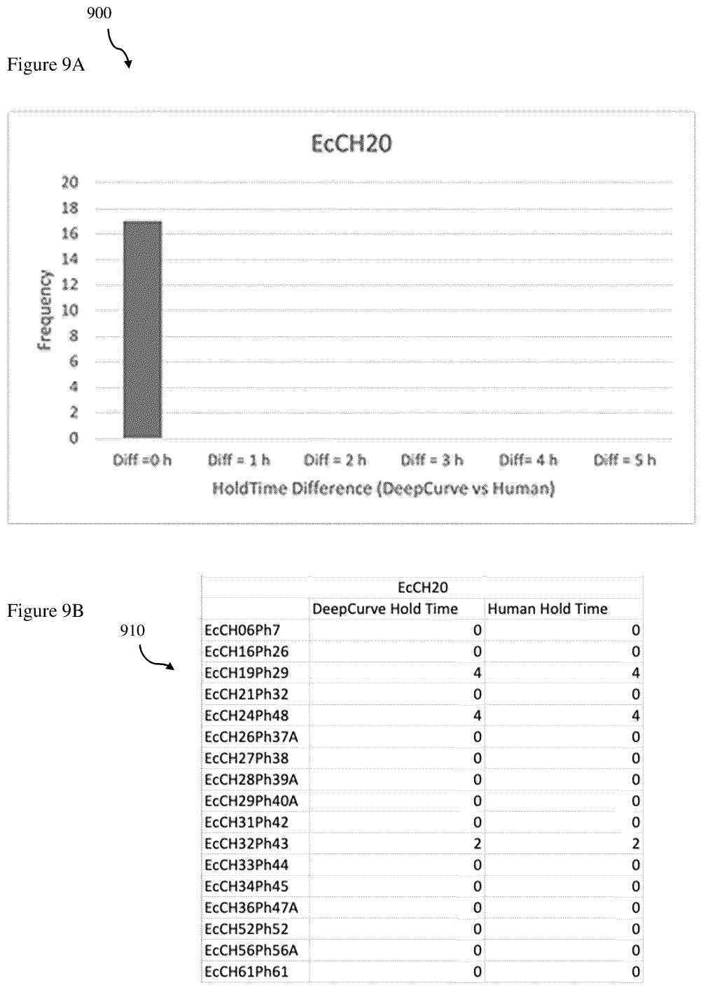

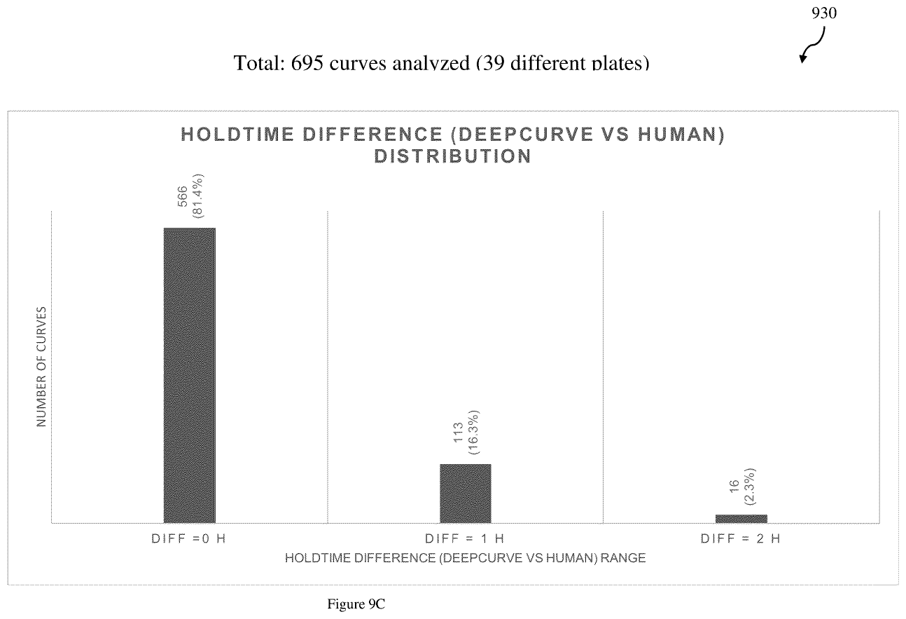

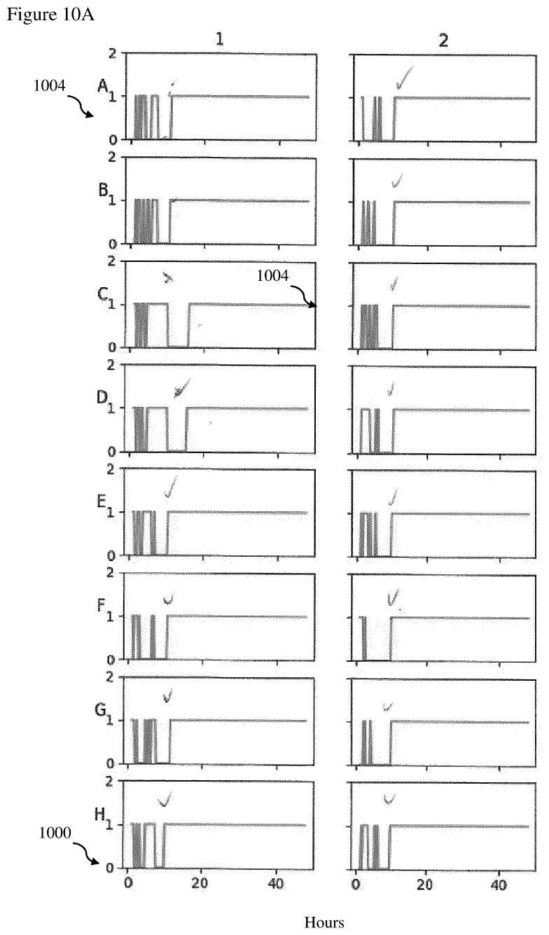

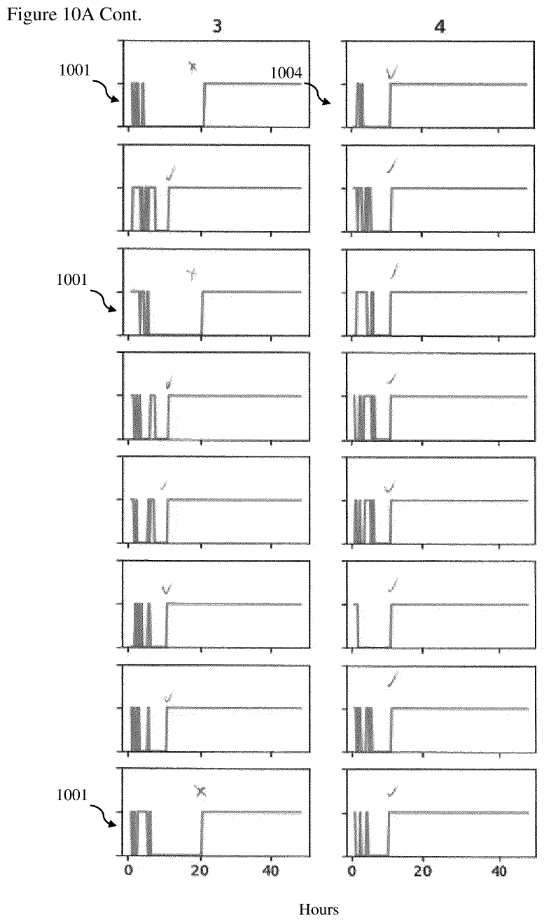

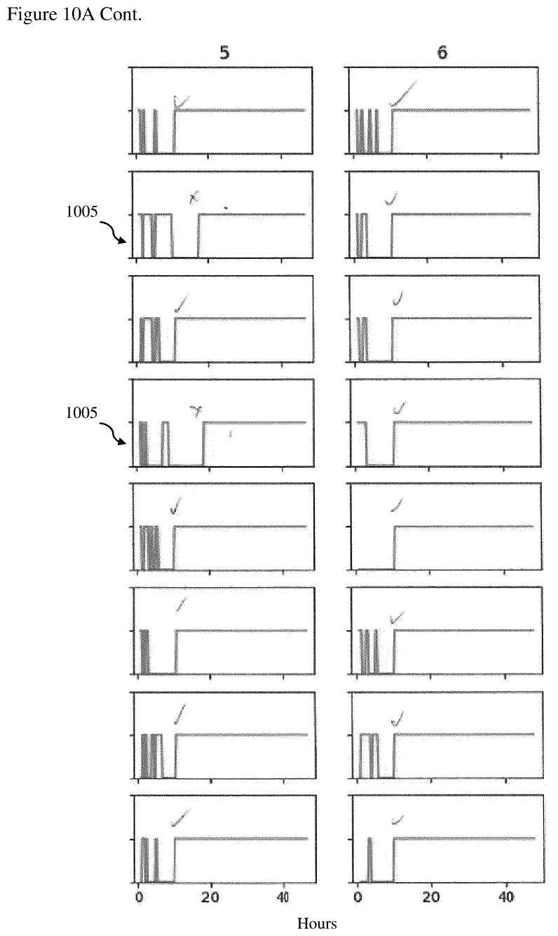

An embodiment of the method was compared to human assessed growth curves and the difference in hold time estimates compared. FIG. 9A is a histogram of the estimated hold time difference between a hold time estimated by an embodiment of the method described herein, and a human estimate and FIG. 9B is a table of the estimated hold time difference between a hold time estimated by an embodiment of the method described herein, and a human estimate used to generate FIG. 9A. These results show that the computer implemented method generated identical results, whilst being considerably faster than the human. Further the precision can be substantially greater than a human in cases where measures are taken more frequently than one every 15 minutes, such as every minute. As more rapid data is obtained humans are unable to distinguish adjacent time points whereas a computer system can analyze each time point. This performance is further illustrated in FIG. 9C is a histogram of the estimated hold time difference between a hold time estimated by an embodiment of the method described herein, and a human estimate for 695 datasets across 39 different multiwell plates. In this larger dataset the model produced identical estimates for 81.4% of cases (curves), no more than a 1 hour difference for 16.5% of cases, and a difference of no more than 2 hours in 2.3% of cases. These results thus show that the computer implemented method is able to robustly fit curves and account for the intrinsic variability of these datasets and produce similar results to an expert human.

The method can thus identify flat curves and robustly fit growth curves. The threshold maximum height h_{TH} may also be used to characterize or classify flat curves, curves with unusual or abnormal growth curves that may appear non-sigmoidal and provisionally classified as flat. However abnormal growth curves typically have substantial variability compared to true flat (i.e., due to the phage inhibiting bacterial growth) and thus can be distinguished by analysis of the variability. This may be determined by performing an analysis of the errors or residuals from a linear fit or other curve fit. A machine learning classifier could also be trained to distinguish between flat curves and abnormal curves.

To examine how quickly the model can distinguish poorly performing phage, an in silico experiment was performed to see how quickly the machine learning model could reliably classify a test host-phage response dataset. In this experiment, a machine learning model was fitted to the complete dataset for each host-phage response, and then a series of test fits was then performed on the dataset where each fit was performed over a progressively increasing time window, using 15-minute intervals. That is a test fit was performed over a time window (0, t) where t was incremented by 15 minutes for each subsequent test fit and the fitted parameters were then provided to the trained machine learning model to provide a classification. As noted above, the trained machine learning model only requires a set of summary parameters, and the time window of the fitted dataset need not be identical to that used to train the model.

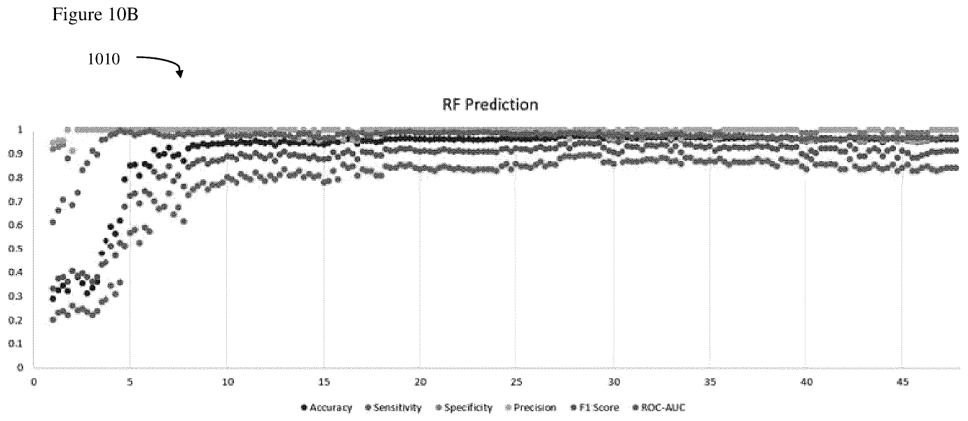

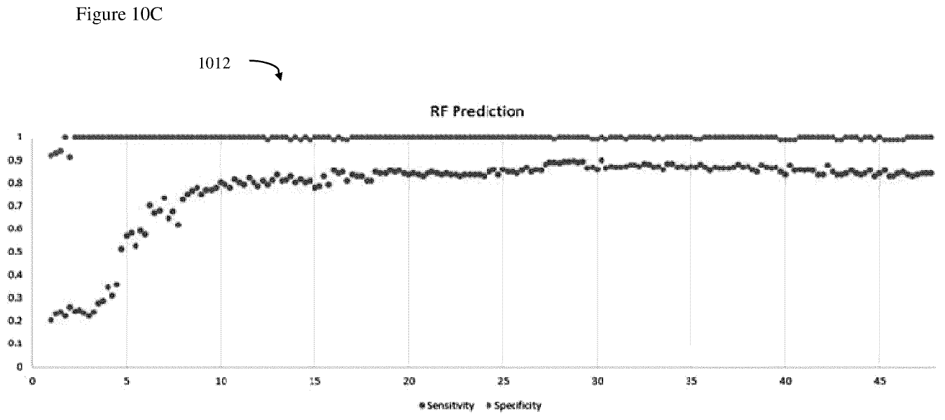

FIG. 10 is a set of 48 plots showing the time taken for a machine learning model to correctly classify the efficacy of a host-phage time series dataset according to an embodiment. Each of the plots shows whether the classification on the test fit agrees with the classification obtained from the machine learning model on the complete dataset, where “1” signifies agreement, and “0” signifies disagreement at each of the 15-minute intervals. Each plot thus shows how long it takes for the machine learning algorithm to generate the correct/stable estimate. Not surprisingly, the plots fluctuate significantly in the first few hours but tend to settle on the correct estimate between 10-20 hours. Notably A3, C3 and H3 are cases where the phage is effective at inhibiting growth, and these each take around 20 hours (time point 1001) for the Machine learning model to make a reliable estimate. This is contrasted with A1, A4 and C2 where the phage is not effective, and these achieve stabile estimates after 10 hours (time point 1004). However, some cells with ineffective phage such as B5 and D5 take longer to stabilize (time point 1005).

These results suggest that the machine learning model is reasonably accurate at quickly predicting poor phage after 10 hours but that it takes longer for effective phage to be identified—in this case around 20 hours). This suggests that the time period should span 20 hours, although tests could be performed after 10 hours to select our clearly ineffective phage. The minimum desirable time period will however depend to some extent on the fitting function used, time window of which fits are performed (e.g., single or piecewise fits), and growth media for the wells used for the host-phage response tests.

Thus, in one embodiment, the method is performed as data is collected and the best fit model used to predict the likely efficacy of the phage, which can then be used to assess whether to stop the experiment before the nominal final or stopping time (e.g. 48 hours). Thus, the method may further comprise receiving an updated host response dataset comprising additional data points (i.e., the current end time point is updated to a later end time point) and repeating the normalization obtaining and reporting steps, wherein the reporting includes an estimate of the probability that the phage is efficacious. This may simply be a re-estimation of the likely class at the current time considering the additional data. In one embodiment the method comprises determining the final class confidence (or final classification expectancy) and reporting this as an estimate of the probability that the phage is efficacious at the current time point. The final class confidence is an estimate of the likelihood that the classification of whether the phage is efficacious at the end time point (i.e., the current class estimate) matches the classification of whether the phage is efficacious at the final time point current (i.e. the final class estimate). The final class confidence could also refer to a final classification expectancy (i.e. the expectancy or likelihood the current class is the same as the final class) or alternatively final class probability (note this is distinct from a class probability which is the confidence in the choice of the current class at the current time point).

The final class confidence may be determined based on identifying a set of similar host phage response datasets in a set of historical host phage response datasets. The final class confidence estimate is determined based on the similar host phage response datasets in which the class at the current end time point matches the final class at the final or stopping time point (e.g., 48 hours) The set of historical datasets used for similarity assessment are full datasets spanning the start time point to the final time point and may include both flat (non-sigmoidal) host phage response datasets and non-flat (sigmoidal) host phage response datasets. In some embodiments the final class and class at each time point may be known. However, these could be re-estimated from the complete dataset if not available, or it is determined it is preferable to use the current classification method such as due to a change in the classification method since classes were originally calculated.

Identifying similar datasets could be performed in several ways. In one embodiment similar historical datasets may be chosen as in those historical datasets which have the same class estimate at the current end time point. In another embodiment similar historical datasets may be chosen as in those historical datasets which have the same class estimate at the current end time point and which match for least a threshold number or percentage (e.g., 75%) of previous time points (e.g. 75%). This could be performed by checking the class at each previous time point and counting the number of matches. In another embodiment correlation measure or a pattern matching methods such as template adaption or signal warping may be used to assess similarity between the two time curves to the current end time point.

In some embodiments the final class confidence may be determined using a machine learning method such as a random forest classifier trained on the historical datasets which can be used generate an estimate of the final class confidence for the current end time point (which can then be reported).

A random forest (RF) based classifier may be trained on the growth curve parameters (Max Height, Slope, Lag, AUC) of both curve types (Flat and Not-Flat). To benchmark the model, a dataset of growth curves was split into two equal subsets, each of them consisting of an equal number of flat and not-flat growth curves. Various parameters such as sensitivity, specificity and accuracy were estimated to assess the predictive power of the ML algorithm. To further test the RF classifier, we estimated the % accuracy of the classifier for each time points from 1 to 20 hr. For each time point between 1 to 20 hours, the RF classifier was trained and tested on the estimated growth curve parameters at that particular time point of various growth curves present in the training and testing set, respectively. The RF classifier is integrated with the analysis module to perform predictions as new data are obtained and it predicts the probability score (ranges between 0 to 1) for every growth curve for each time step from 1 to 20 hr. If the predicted score is >=0.50, the growth curve is classified as Not-Flat (Bad Phage) and if the probability score is <0.50, the growth curve is classified as Flat (Good Phage).

FIG. 10B is a plot 1010 of the various performance measures of random forest predictor for Acinetobacter baumannii over time and FIG. 10C is a plot 2012 showing sensitivity and specificity. Similar performance is observed for other bacteria such as Klebsiella pneumoniae and Escherichia coli.

A range of machine learning classifiers may be used, such as a Boosted Trees Classifier, Random Forest Classifier, Decision Tree Classifier, Support Vector Machine (SVM) Classifier, Logistic Classifier, etc. In some embodiments the classifier is a probabilistic classifier. That is rather than just issuing a binary classification (e.g., efficacious or not), the classifier outputs the class probability. Probabilistic classifiers include naïve Bayes, binomial regression models, discrete choice models, decision tree and boosting based classifiers. In some embodiments machine learning training comprises separating the complete dataset into a first training dataset and a second validation dataset. The training dataset is preferably around 60-80% of the total dataset. This dataset is used by machine learning models to create a classifier model to accurately identify efficacious phage. The second set is the Validation dataset, which is typically at least 10% of the dataset and more preferably 20-40%: This dataset is used to validate the accuracy of the model created using the training dataset. Data may be randomly allocated to the training dataset and validation datasets. In some embodiments, checks may be performed on the training dataset and validation datasets to ensure a similar proportion of good/bad phage are present in each. In some embodiments where cross-validation is used the dataset may be allocated to three datasets, namely a training dataset, a validation dataset, and a holdout or test dataset. The third holdout or test dataset is typically around 10-20% of the total dataset and is not used for training the machine learning classifier or the cross validation. This holdout dataset provides an unbiased estimate of the accuracy of the machine learning classifier model.

In one embodiment, the final class confidence estimate may be used to make decisions on whether to continue an experiment for a specific phage host combination or entire plate. For example, if the final class confidence exceeds a stopping threshold (e.g., 67%, 75%, 90%, 95% or 99%) the user can have confidence the final (correct) class has been determined, and the experiment/assay for that host-phage combination can be stopped. This allows another experiment/assay using a new host-phage combination to be started earlier than would normally occur, or it may allow treatment to be initiated using the phage (or phage-host combination) with the high final class confidence. In some embodiments the plate may contain many wells (e.g., 96) and it may not be desirable to continue the experiment until a threshold number (e.g., 75%), or all, of the wells have final class confidence over the stopping criteria. Phage host combinations/wells which were terminated early and which had final class confidence estimate below the stopping threshold could be restarted. In some embodiments specialized plates may be constructed to allow swapping mid experiment/assay. For example, a 96 well plate could comprise 12 swappable 8×1 columns of wells.

In one embodiment, the fitting step could be performed repeatedly during the course of the host phage experiment. That is, as the experiment progresses, and further data becomes available, the dataset is updated with the additional data points (i.e., the additional times) and the fitting function is refitted and classified on the updated dataset. This is equivalent to progressively increasing the time window with each new fit. In another embodiment the width of the fitting time window could be fixed such that the fitting process is effectively using a sliding time window as further data becomes available. In these embodiments, a probabilistic classifier may be used to output the classification probability. Alternatively, a classification expectancy could be estimated with each new time point/fit. The classification expectancy is an estimate of the probability (or likelihood) that the classification result being correct conditional on the current state determined using the distributions of historical data which contain a point matching the current state at the current time. That is, given set of parameters at a given time in the assay, a number could be produced that is measure of the confidence of the classification outcome (i.e., is the current classification result the expected result) for a given phage. For example, new data could be obtained every 15 minutes, and the classifiers decision could be saved for each time point.

To obtain the classification expectancy at each point we extract the subset of the historical dataset that had a matching current state. In a first embodiment this could be the dataset with the same classification outcome at the current time point. Having obtained this subset we then determine the percentage of the subset where the current estimate of the classification result was the same as the final classification result (e.g., the classification after completion of the assay) and we return that percentage (or a number based on that percentage). As time progresses this is expected to stabilize on the final value. That is, for an assay performed over 24 hours, we may get a classification result at 4 hours with a probability of 50% (i.e., unstable estimate). By 12 hours the probability may be 75% (likely to be accurate), and by 20 hours it may be 99% (highly likely to be accurate). In another embodiment, the dataset could be the dataset with the same classification outcome at the current time point and with growth measure within some range of the observed growth measure at the current time. This could be achieved by partitioning the growth values into a set of intervals or bins (e.g., 0 to 0.1, 0.1 to 0.2, 0.3 to 0.4, etc.).

We then identify which interval/bin the observed growth measure falls within and select the subset of historical data that had observed growth measures in the same interval/bin at the same time with the same current classification result. Having obtained this subset, we then determine the percentage of the subset where the current classification result was the same as the final classification result (i.e., is the current classification result the expected classification result). In an alternative embodiment, the dataset could be the dataset with the growth measure within some range of the observed growth measure at the current time as described above (i.e., selection of the dataset ignores the current classification result). We then return the percentage of final classification result for this subset which had a final classification result that matches the current classification result. The classification expectancy can thus provide an early measure of the confidence or stability of the current classification result by leverages the longer time series (and outcomes) available in a historical dataset.

The above embodiments can be used to identify one or more efficacious phage for a host bacterium. Where multiple phages are tested against the same host bacteria, a therapeutic phage formulation of the most effective phage(s) can be generated for treatment or some other criteria such as inventory status. Selection of which phage, or phages, to include may be obtained using a measure of diversity of the efficacious phage. In one embodiment the measure of diversity is indicative of a different mechanism of action between the phages. This measure of diversity could be estimated by sequencing the phage and using bioinformatics methods or datasets to estimate functional effects/associations and these could be used to assign one or more mechanism of actions labels (these could be selected from a controlled ontology such as the GeneOntology database, or databases of biological networks). Phage combinations can thus be selected based on those with different mechanisms of actions, or where phage are assigned a set of multiple possible mechanisms of action, phage could be selected based on the two phage with most dissimilar sets (i.e. minimum overlap of possible mechanisms of action). Overlapping methods of actions could be defined based on sharing a biological network or pathway, or a GeneOntology (GO) term (or downstream of a GO term), or a GO-CAM model. For example, each pair of phage could be assigned a score based on the number of mechanism of actions not shared by both lists. The largest score would indicate the most diverse (non overlapping) list. In another example, the score could be a weighted score. For example, the previous score could be divided by sum of the two list sizes to weight for list size. Other weighting or scoring functions could be used, such as applying a weighting that takes into account the evidence for a mechanism of action associated with a sequence. Other methods of assessing diversity of possible mechanisms of action could also be used based on bioinformatics data mining or biological network/pathway analysis. This approach provides robustness against a bacterium adapting to the mechanism of action of a single phage, as if the second phage has a different mechanism of action then it is likely to remain effective.

Embodiments described herein thus advantageously provide automated methods for analyzing/interpreting host phage response data that are designed to be robust to the intrinsic variability observed in host phage response datasets. Embodiments of the method fits functions over a range of time points from the start time to an end time and at each time point uses a sequential fitting approach using a sequence of sigmoidal functions such as Gompertz, Logistic and Richards functions (although others could be used). The best fit time point and model parameters can thus be obtained from the fit, for example to obtain the lag time from which a hold time can be obtained. The method gains robustness by using several different fitting functions to allow the method to adapt to the substantial variability and experimental effects observed in host phage response datasets.

Further to prevent the fitting from focusing on biologically non-interesting time periods such as the early lag phase or late stable phase and an adjustment step is performed to search for fits of similar quality (e.g., based on similar R²) but closer to the minimum lag time. That is in return for potentially sacrificing some quality/goodness of fit the method drives the estimate of the best time towards more central, and biologically interesting results.

A fully loaded OmniLog™ apparatus contains multiple multi-well plates and takes about 15 minutes to scan every well in the apparatus. The fitting process is rapid and can thus be performed after each well or after each plate is read and a report of results generated, such as the color-coded matrix plot shown in FIGS. 8b and 8C. Further prediction of the likely classification can be determined as the experiment progresses.

The automated nature saves considerable manual labor and time, with a pair of human experts taking around 45 minutes to analyze 48 plates. In contrast embodiments of the method can analyze 48 plates in around 10 minutes. Further the method is robust and results can be automatically exported to a data storage, web server, or laboratory information management system, along with associated experimental meta data.

The approach can be used to identify phage for including in phage formations for treating patients with bacterial infections, and in particular Multiple Drug Resistant Infections. The methods can also be used to identify phage that can be used to clean up bacterial contaminated areas, such as for cleaning up an industrial site. These phage formulations may include two or more phage with different mechanisms of action as described above.

Variations on the above methods can also be performed. In one embodiment, the historical dataset is used to improve classification when performed during the assay (i.e., at some time point before the full assay time period). In this embodiment, a fit (or multiple fits) is performed over the current time period (e.g., 0 to 6 hours). Then fit results over the same time period is obtained for each host-phage profile in an historical dataset is obtained, and a subset of the historical dataset is selected based on having fit results similar to the fit results for the current host-phage combination (over the current time period). That is, we identify the subset of the historical dataset with a similar phage-host curve to the observed phage-host curve up to this point in time (or over some time range to this point in time). Determining similar phage-host curves could be performed using correlation measures (e.g., a cross correlation or similar similarity measure). We then provide additional data from the historical dataset as further inputs to the classifier (beyond just the fit values). In one embodiment this might be percentage of this subset of the historical dataset which were ultimately efficacious. In one embodiment a random forest classified may be used

FIG. 3 depicts an exemplary computing system configured to perform any one of the computers implemented methods described herein. The computing system may comprise one or more processors including multi-core CPUs and Graphical Processing Units (GPUs) operatively connected to one or more memories which store instructions to configure the processor to perform embodiments of the method. In this context, the computing system may include, for example, one or more processors (CPUs, GPUs), memories, storage, and input/output devices (e.g., monitor, keyboard, disk drive, network interface, Internet connection, etc.). However, the computing system may include circuitry or other specialized hardware for carrying out some or all aspects of the processes. The computing system may be a computing apparatus such as an all-in-one computer, desktop computer, laptop, tablet or mobile computing apparatus, server, and any associated peripheral devices. The computer system may be a distributed system including server-based systems and cloud-based computing systems. In some operational settings, the computing system may be configured as a system that includes one or more units, each of which is configured to carry out some aspects of the processes either in software, hardware, or some combination thereof. For example, the user interface may be provided on a desktop computer or tablet computer, whilst the fitting may be performed on a server-based system including cloud based server systems, and the user interface is configured to communicate with such servers. The user interface may be provided as a web portal, allowing a user on one computer to upload datasets which may be processed on a remote computing apparatus or system (e.g., server or cloud system) and which provides the results (i.e., the report) back to the user, or to other users on other computing apparatus.

FIG. 3 depicts an exemplary computing system (300) with a number of components that may be used to perform the processes described herein. For example, an input/output (“I/O”) interface 330, one or more central processing units (“CPU”) (340), and a memory section (350). The I/O interface (330) is connected to input and output devices such as a display (320), a keyboard (310), a disk storage unit (390), and a media drive unit (360). The media drive unit (360) can read/write a computer-readable medium (370), which can contain programs (380) and/or data. The I/O interface may comprise a network interface and/or communications module for communicating with an equivalent communications module in another device using a predefined communications protocol (e.g., Bluetooth, Zigbee, IEEE 802.15, IEEE 802.11, TCP/IP, UDP, etc.).

A computer program may be written, for example, in a general-purpose programming language (e.g., Pascal, C, C++, Java, Python, JSON, etc.) or some specialized application-specific language to provide a user interface, perform the model fitting, and export results. In one embodiment the machine learning model may be generated using a machine learning libraries/packages such as SciKit-Learn, Tensorflow, and PyTorch, may be used. These typically implement a plurality of different classifiers such as a Boosted Trees Classifier, Random Forest Classifier, Decision Tree Classifier, Support Vector Machine (SVM) Classifier, Logistic Classifier, etc. These can each be tested, and the best performing classifier selected.

The steps of a method or algorithm described in connection with the embodiments disclosed herein may be embodied directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors including CPU and GPUs, controllers, micro-controllers, microprocessors, other electronic units designed to perform the functions described herein, or a combination thereof. Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

A non-transitory computer-program product or storage medium comprising computer-executable instructions for carrying out any of the methods described herein can also be generated. A non-transitory computer-readable medium can be used to store (e.g., tangibly embody) one or more computer programs for performing any one of the above-described processes by means of a computer. Further provided is a computer system comprising one or more processors, memory, and one or more programs, wherein the one or more programs are stored in the memory and configured to be executed by the one or more processors, the one or more programs including instructions for carrying out any of the methods described herein.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A computer implemented method for analyzing host phage response data, the method comprising:
receiving or accessing a host phage response dataset, wherein the dataset comprises a time series dataset for a host-phage combination in which a host bacteria is grown in the presence of a phage, and each data point in the time series dataset comprises a measurement of a parameter indicative of the growth of the host bacteria in the presence of the phage at a specific time;
obtaining an estimate of a lag time comprising:
for a plurality of time points in a time window from an initial time point to an end time point, fitting one or more candidate functions over a fitting time window from the initial time point to a current time point, wherein fitting estimates a set of growth curve summary parameters comprising at least a lag time and a goodness of fit parameter;
selecting a best fit function for the current time point from the one or more fitted candidate functions based on the goodness of fit parameters, wherein the one or more candidate functions each comprise a different functional form and at least one of which is a sigmoidal function, and when none of the goodness of fit estimates pass a threshold goodness of fit then classifying the time series dataset as flat and setting the lag time to the end time point;
selecting from the plurality of time points, a best time point based on goodness of fit values at the plurality of time points;
searching for an alternative best time point closer in time to, and greater than, a minimum time point than the best time fit and updating the best time point to the alternative best time point when the goodness of fit of the alternative time point is within a threshold amount of the goodness of fit of the best time point;
estimating a lag time for the best time point, wherein when the goodness of fit exceeds a threshold goodness of fit the lag time is obtained from the growth curve summary parameter otherwise classifying the time series dataset as flat and setting the lag time to the end time point; and
reporting at least the lag time or a hold time calculated as the estimated lag time from which the lag time for a control is subtracted.

2. The computer implemented method as claimed in claim 1, wherein the one or more candidate functions is an ordered set of candidate functions, and fitting one or more candidate functions comprises sequentially fitting each candidate function according to an order in the ordered set and the sequential fitting is terminated and the candidate function is selected as the best fit function when the goodness of fit of the fitted candidate function exceeds a predefined threshold goodness.

3. The computer implemented method as claimed in claim 2, wherein the ordered set of candidate functions comprises a Gompertz function, a Logistic function and a Richards function.

4. The computer implemented method as claimed in claim 3, wherein when fitting of each of the Gompertz function, the Logistic function and the Richards function failed to generate a goodness of fit exceeding the predefined threshold goodness, then applying a Blackman window function to the time series data and repeating the fitting process, and when the repeated fits for each of the Gompertz function, the Logistic function and the Richards function fail to generate a goodness of fit exceeding the predefined threshold goodness, then classifying the time series dataset as flat and setting the lag time to the end time point.

5. The computer implemented method as claimed in claim 1, wherein prior to fitting one or more candidate functions over a fitting time window, determining a maximum height of the time series dataset, and when the maximum height is less than a threshold maximum height, then classifying the time series dataset as flat and setting the lag time to the end time point and terminating the fitting process.

6. The computer implemented method as claimed in claim 1, wherein searching for an alternative best time point comprises:
determining when the best time point is less than the minimum time point and when the best time point is less than the minimum time point then the alternative time point is selected based on the closest alternative time point on or after the minimum time point with a goodness of fit within a threshold difference of the goodness of fit for the best time point, and
when the best time point is greater than the minimum time point then the alternative time point is selected based on the alternative time point being greater than or equal to the minimum time point and which is the closest alternative time point to the minimum time point and with a goodness of fit within a threshold difference of the goodness of fit for the best time point.

7. The computer implemented method as claimed in claim 6, wherein the minimum time point is 5 hours, the goodness of fit is the coefficient of determination (R²), and the threshold difference is 0.03.

8. The computer implemented method as claimed in claim 6, wherein the goodness of fit is the co-efficient of determination (R²) and the predefined threshold goodness is 0.6.

9. The computer implemented method as claimed in claim 1, wherein the end time point is 48 hours.

10. The computer implemented method as claimed in claim 1, wherein the minimum time point is 5 hours.

11. The computer implemented method as claimed in claim 1, wherein when the time series data is classified as flat, estimating a variability measure and when the variability measure exceeds a variability threshold rejecting the time series dataset and classifying the time series dataset as abnormal.

12. The computer implemented method as claimed in claim 1, further comprising normalizing the time series dataset based on an associated control curve.

13. The computer implemented method as claimed in claim 12, wherein a host-growth curve is a host only time-series dataset and normalizing the time series dataset comprises subtracting the host only time-series dataset from the time series dataset, wherein the time series dataset and the host only time-series dataset are obtained from separate wells on a same multi-well plate.

14. The computer implemented method as claimed in claim 13, wherein the multi-well plate further comprises one or more media control wells and the computer implemented method further comprises performing quality assurance comprising at least identifying anomalous media control wells or anomalous host cell only wells and excluding time-series datasets associated with any identified anomalous media control wells or anomalous host cell wells.

15. The computer implemented method as claimed in claim 1, wherein the time series dataset is obtained from a multi-well plate comprising a plurality of host-phage combinations, a set of positive control wells, a set of media control wells, a set of host cell control wells, a first set of diluted host cell wells and a second set of diluted host cell wells, and the computer implemented method is performed for each host-phage combination on the multi-well plate, and a report is generated for each host-phage combination on the multi-well plate.

16. The computer implemented method as claimed in claim 1, wherein the growth curve summary parameters comprise at least a max height, a slope, a lag time, and an area under curve, and the goodness of fit comprises one or more of a coefficient of determination (R²), a parameter based upon an error term or a residual term, or a summary statistic of residuals.

17. The computer implemented method as claimed in claim 12, wherein the end time point is prior to a final time point and the method further comprising receiving an updated host response dataset comprising additional data points and repeating the normalization obtaining and reporting steps, wherein the reporting includes an estimate of a probability that the phage is efficacious.

18. The computer implemented method as claimed in claim 1, wherein the end time point is prior to a final time point, further comprising determining a final class confidence estimate which is an estimate that a classification of whether the phage is efficacious at the end time point matches the classification of whether the phage is efficacious at the final time point and reporting the estimate that the phage is efficacious further comprises reporting the estimate that the phage is efficacious at the end point and final class confidence estimate, wherein determining a final class confidence estimate is determined based on identifying a set of similar host phage response datasets in a set of historical host phage response datasets comprising a plurality of flat host phage response datasets and a plurality of non-flat host phage response datasets and each comprising data points from a start time to the final time, wherein the final class confidence estimate is determined based on the similar host phage response datasets in which an estimate that the classification of whether the phage is efficacious at the end time point matches the classification of whether the phage is efficacious at the final time point.

19. The computer implemented method as claimed in claim 18, wherein the final class confidence estimate is generated using a random forest based classifier trained on the set of historical host phage response datasets.

20. The computer implemented method as claimed in claim 18, wherein a phage is selected based on the final class confidence estimate exceeding a stopping threshold at an end time point prior to the final time point.

21. A non-transitory, computer program product comprising computer executable instructions for analyzing host phage response data, the instructions executable by a computer for:
receiving a host phage response dataset, wherein the dataset comprises a time series dataset for a host-phage combination in which a host bacteria is grown in the presence of a phage, and each data point in the time series dataset comprises a measurement of a parameter indicative of the growth of the host bacteria in the presence of the phage at a specific time;
normalizing the time series dataset based on an associated control curve;
obtaining an estimate of a lag time comprising:
for a plurality of time points in a time window from an initial time point to an end time point, fitting one or more candidate functions over a fitting time window from the initial time point to a current time point, wherein fitting estimates a set of growth curve summary parameters comprising at least a lag time and a goodness of fit parameter;
selecting a best fit function for the current time point from the one or more fitted candidate functions based on the goodness of fit parameters, wherein the one or more candidate functions each comprise a different functional form and at least one of which is a sigmoidal function, and when none of the goodness of fit estimates pass a threshold goodness of fit then classifying the time series dataset as flat and setting the lag time to the end time point;
selecting from the plurality of time points, a best time point based on goodness of fit values at the plurality of time points;
searching for an alternative best time point closer in time to, and greater than, a minimum time point than the best time fit and updating the best time point to the alternative best time point when the goodness of fit of the alternative time point is within a threshold amount of the goodness of fit of the best time point; and
estimating a lag time for the best time point, wherein when the goodness of fit exceeds a threshold goodness of fit the lag time is obtained from the growth curve summary parameter otherwise classifying the time series dataset as flat and setting the lag time to the end time point; and
reporting at least the lag time or a hold time calculated as the estimated lag time from which the lag time for a control is subtracted.

22. A computing apparatus comprising:
at least one memory, and
at least one processor wherein the memory comprises instructions to configure the at least one processor for:
receiving a host phage response dataset, wherein the dataset comprises a time series dataset for a host-phage combination in which a host bacteria is grown in the presence of a phage, and each data point in the time series dataset comprises a measurement of a parameter indicative of the growth of the host bacteria in the presence of the phage at a specific time;
normalizing the time series dataset based on an associated control curve;
obtaining an estimate of a lag time comprising:
for a plurality of time points in a time window from an initial time point to an end time point, fitting one or more candidate functions over a fitting time window from the initial time point to a current time point, wherein fitting estimates a set of growth curve summary parameters comprising at least a lag time and a goodness of fit parameter;
selecting a best fit function for the current time point from the one or more fitted candidate functions based on the goodness of fit parameters, wherein the one or more candidate functions each comprise a different functional form and at least one of which is a sigmoidal function, and when none of the goodness of fit estimates pass a threshold goodness of fit then classifying the time series dataset as flat and setting the lag time to the end time point;
selecting from the plurality of time points, a best time point based on goodness of fit values at the plurality of time points;
searching for an alternative best time point closer in time to, and greater than, a minimum time point than the best time fit and updating the best time point to the alternative best time point when the goodness of fit of the alternative time point is within a threshold amount of the goodness of fit of the best time point; and
estimating a lag time for the best time point, wherein when the goodness of fit exceeds a threshold goodness of fit the lag time is obtained from the growth curve summary parameter otherwise classifying the time series dataset as flat and setting the lag time to the end time point; and
reporting at least the lag time or a hold time calculated as the estimated lag time from which the lag time for a control is subtracted.

23. The computer implemented method of claim 1, wherein the host bacteria is grown as planktonic cells.

24. The computer implemented method of claim 1, wherein the host bacteria is grown as a biofilm.

25. The computer implemented method of claim 1, wherein phage-phage synergy, antibiotic-phage synergy, or antibiotic-phage-phage synergy is measured.

26. The non-transitory, computer program product of claim 21, wherein the host bacteria is grown as planktonic cells.

27. The non-transitory, computer program product of claim 21, wherein the host bacteria is grown as a biofilm.

28. The non-transitory, computer program product of claim 21, wherein phage-phage synergy, antibiotic-phage synergy, or antibiotic-phage-phage synergy is measured.

29. The computing apparatus of claim 22, wherein the host bacteria is grown as planktonic cells.

30. The computing apparatus of claim 22, wherein the host bacteria is grown as a biofilm.

31. The computing apparatus of claim 22, wherein phage-phage synergy, antibiotic-phage synergy, or antibiotic-phage-phage synergy is measured.
